(19) United States
(12) Patent Application Publication
(10) Pub. No.: US 2015/0217100 A1
(43) Pub. Date: Aug. 6, 2015

(54) URETHRAL STRICTURE TREATMENT METHOD

(71) Applicant: TERUMO KABUSHIKI KAISHA, Shibuya-ku (JP)

(72) Inventors: Wataru KARINO, Atsugi-shi (JP); Riyaheh ARASTOO, Hadano-shi (JP); Kosuke NISHIO, Machida-shi (JP)

(73) Assignee: TERUMO KABUSHIKI KAISHA, Tokyo (JP)

(21) Appl. No.: 14/611,473

(22) Filed: Feb. 2, 2015

(30) Foreign Application Priority Data
Feb. 3, 2014 (JP) 2014-018965

(51) Int. Cl. A61M 37/00; A61B 17/22; A61B 1/307; A61B 17/3209

(52) U.S. Cl. CPC A61M 37/00; A61B 17/3209; A61B 17/22; A61B 1/307

(57) ABSTRACT

A urethral stricture treatment method is disclosed that inhibits recurrence of urethral stricture by imparting an epithelial function to a treatment portion formed by treating scar tissue. The urethral stricture treatment method can include a treatment step of treating scar tissue by using a treatment tool transurethrally inserted into a urethra and forming a treatment portion to which an epithelial function can be imparted, a confirmation step of confirming whether or not the treatment portion has been formed by using a diagnostic tool transurethrally inserted into the urethra, and an assisting step of imparting the epithelial function to the treatment portion by using an assisting means transurethrally inserted into the urethra.

### CROSS-REFERENCES TO RELATED APPLICATIONS

This application claims priority to 35 U.S.C. §119 to Japanese Patent Application No. 2014-018965 filed in Japan on Feb. 3, 2014, the entire content of which is incorporated herein by reference.

### TECHNICAL FIELD

The present disclosure generally relates to a urethral stricture treatment method.

### BACKGROUND DISCUSSION

Urethral stricture is narrowing of the urethra that is a disease caused by the following mechanism. When the urethral mucosa is wounded by injury or inflammation, the urethral mucosa or the corpus spongiosum penis surrounding the urethral mucosa undergoes cicatrization in the process of wound healing, and as a result, the urethra is narrowed. As a urethral stricture treatment method, there is a method of surgically reconstructing the urethra. However, the method can be highly invasive and can require an extended period of hospitalization. Therefore, in recent years, an endoscopic dilation procedure using a less invasive and simple bougie (urethral dilator) or a balloon catheter described in Japanese Patent Application No. 8-526731 that can be used in the urethra, a cold knife, a laser, and the like has been performed.

### SUMMARY

However, the aforementioned treatment method of the prior art is practically incapable of reconstructing epithelial cells on the inner surface of the urethra having turned into scar tissue. Accordingly, the method can have problems in that it results in the recurrence of the disease and can cause endless suffering to patients. For example, because urine can permeate the scar tissue, an inflammatory reaction can continuously occur in the urethra, and thus the tissue is thickened. Consequentially, reocculusion is induced, and which can be considered a main cause of the recurrence of the disease.

In accordance with an exemplary embodiment, a urethral stricture treatment method is disclosed that can inhibit the recurrence of urethral stricture by treating scar tissue and imparting an epithelial function to the thus formed treatment portion.

In accordance with an exemplary embodiment, the urethral stricture treatment method of the present disclosure can include a treatment step of treating scar tissue by a treatment tool transurethrally inserted into the urethra and forming a treatment portion to which an epithelial function can be imparted, a confirmation step of confirming whether or not the treatment portion has been formed by using a diagnosis tool transurethrally inserted into the urethra, and an assisting step of imparting the epithelial function to the treatment portion by using assisting means transurethrally inserted into the urethra.

According to the present disclosure, a treatment portion can be formed by performing a predetermined treatment on scar tissue causing urethral stricture, and then an epithelial function is imparted to the treatment portion. In this way, the occurrence of restenosis caused by thickening of the scar tissue can be inhibited. Moreover, because each of the treatment step for forming a treatment portion, the confirmation step for confirming whether or not the treatment portion has been formed, and the assisting step for imparting an epithelial function to the treatment portion is transurethrally performed, a less invasive procedure that greatly reduces the strain imposed on a patient can be realized.

Furthermore, if the confirmation step includes a step of confirming whether or not at least a portion of the corpus spongiosum penis forming the treatment portion has been exposed from the scar tissue based on a diagnostic image captured by an imaging apparatus, and the treatment in the treatment step is performed until the corpus spongiosum penis is confirmed, the corpus spongiosum penis from being unnecessarily damaged in the treatment step can be prevented.

In addition, if the confirmation step includes a step of visually determining whether or not the corpus spongiosum penis has been exposed based on the color change of the diagnostic image, the exposure of the corpus spongiosum penis can be visually confirmed in a relatively simple way and a more accurate determination can be made.

Moreover, if the treatment step includes a step of making an incision in the scar tissue, the treatment portion can be transurethrally formed in a relatively simple and rapid way.

Furthermore, if the treatment step includes a step of scraping the scar tissue, the treatment portion can be transurethrally formed in a relatively simple and rapid way.

In addition, if the treatment step includes a step of performing the treatment plural times while changing the depth stepwise in the thickness direction of the scar tissue, the corpus spongiosum penis can be reliably prevented from being unnecessarily damaged during the formation of the treatment portion.

In addition, if the treatment further includes a depth diagnosing step of diagnosing the depth of the treatment performed on the scar tissue, the treatment can be performed to an appropriate depth. Accordingly, the corpus spongiosum penis can more reliably be prevented from being damaged during the formation of the treatment portion.

Furthermore, if the treatment step includes a step of performing the treatment on the scar tissue while avoiding a direction in which the corpus cavernosum penis is positioned in the circumferential direction of the urethra, the corpus cavernosum penis can be prevented from being damaged during the formation of the treatment portion.

Moreover, if the assisting means is not infiltrated by liquid, the reconstruction of the epithelial function in the treatment portion can be suitably assisted.

In addition, if the assisting means includes the epithelium collected from a biological body, epithelial cells can be engrafted into the treatment portion. Accordingly, the occurrence of restenosis can be more reliably prevented.

In accordance with an exemplary embodiment, a urethral stricture treatment method is disclosed comprising: a treatment step of performing a treatment on scar tissue by using a treatment tool transurethrally inserted into a urethra and forming a treatment portion to which an epithelial function can be imparted; a confirmation step of confirming whether or not the treatment portion has been formed by using a diagnostic tool transurethrally inserted into the urethra; and an assisting step of imparting the epithelial function to the treatment portion by using an assisting means transurethrally inserted into the urethra.

In accordance with an exemplary embodiment, a method of treating a urethral stricture is disclosed, the method comprising: performing a treatment on scar tissue by using a treatment tool transurethrally inserted into a urethra and forming a treatment portion to which an epithelial function can be imparted; confirming whether or not the treatment portion has been formed by using a diagnostic tool transurethrally inserted into the urethra; and imparting the epithelial function to the treatment portion by using an assisting means transurethrally inserted into the urethra.

### BRIEF DESCRIPTION OF DRAWINGS

FIGS. 1A and 1B are views showing the urethra of a biological body to which a treatment method according to an embodiment is applied, wherein FIG. 1A is a view schematically showing the periphery of the urethra of the biological body, and FIG. 1B is a cross-sectional view of the urethra taken along line 1B-1B shown in FIG. 1A.

FIG. 2 is a cross-sectional view of the urethra and the periphery thereof shown in FIGS. 1A and 1B that is taken along line 2-2 (line running along the direction in which the urethra extends) shown in FIG. 1A.

FIGS. 3A and 3B are enlarged views showing the urethra and corpus spongiosum penis, wherein FIG. 3A is an enlarged cross-sectional view of the periphery of the urethra shown in FIG. 1B, and FIG. 3B is an enlarged cross-sectional view of a portion 3B indicated by a dotted line in FIG. 3A.

FIG. 4 is a view showing the flow of the respective steps of the treatment method according to the embodiment.

FIG. 5 is a schematic perspective view of a treatment tool used in a treatment step.

FIGS. 6A and 6B are views for illustrating the constitution of the treatment tool shown in FIG. 5, wherein FIG. 6A is a cross-sectional view of a tip portion of the treatment tool accommodating a blade portion that is taken along the longitudinal direction of the treatment tool, and FIG. 6B is a cross-sectional view taken along line 6B-6B shown in FIG. 6A.

FIGS. 7A and 7B are views for illustrating the constitution of the treatment tool shown in FIG. 5, wherein FIG. 7A is a cross-sectional view of the distal end portion of the treatment tool from which the blade portion has protruded that is taken along the longitudinal direction of the treatment tool, and FIG. 7B is a cross-sectional view taken along line 7B-7B shown in FIG. 7A.

FIGS. 8A and 8B are views for illustrating the treatment step according to the embodiment, wherein FIG. 8A is a cross-sectional view showing a state in which the treatment tool has been inserted into the urethra, and FIG. 8B is a cross-sectional view showing a state in which the treatment step is performed by using the treatment tool inserted into the urethra.

FIGS. 9A and 9B are views for illustrating the treatment step and a confirmation step according to the embodiment, wherein FIG. 9A is a cross-sectional view showing a state in which the treatment step is performed by using the treatment tool inserted into the urethra, and FIG. 9B is a cross-sectional view showing a state in which the confirmation step is performed by using the treatment tool inserted into the urethra.

FIGS. 10A to 10C are views showing treatment tools according to Modification examples 1 to 3.

FIGS. 11A and 11B are views for illustrating the treatment step of forming a treatment portion by a treatment of scraping scar tissue, wherein FIG. 11A is a cross-sectional view showing a state in which the treatment step is performed by using the treatment tool inserted into the urethra, and FIG. 11B is a cross-sectional view showing the treatment portion formed by the treatment step shown in FIG. 11A.

FIGS. 12A and 12B are cross-sectional views showing main portions of treatment tools according to Modification examples 4 and 5.

FIGS. 13A to 13C are views showing treatment tools according to Modification examples 6 to 8.

FIGS. 14A to 14C are partial cross-sectional views showing the constitution of a treatment tool according to Modification example 9 and showing an example of how to use the treatment tool.

FIGS. 15A to 15C are partial cross-sectional views showing an example of how to use the treatment tool according to Modification example 9.

FIGS. 16A and 16B are views for illustrating a smoothening step according to an embodiment, wherein FIG. 16A is a cross-sectional view showing the urethra of which the inner surface has been smoothened by a predetermined smoothening tool, and FIG. 16B is an enlarged cross-sectional view showing a portion of FIG. 16A.

FIGS. 17A and 17B are views showing an example of how to use the smoothening tools according to Modification examples 1 and 2.

FIGS. 18A and 18B are views illustrating a hemostatic step according to an embodiment, wherein FIG. 18A is a cross-sectional view showing a state in which hemorrhage occurring in the vicinity of the treatment portion has been stopped by a predetermined hemostatic tool, and FIG. 18B is a cross-sectional view showing an example of how to use a hemostatic tool according to Modification example 1.

FIGS. 19A and 19B are views showing hemostatic tools according to Modification examples 2 and 3.

FIGS. 20A to 20C are partial cross-sectional views showing an example of how to use the hemostatic tool according to Modification example 2.

FIGS. 21A to 21C are views showing an insertion tool used in an insertion step according to an embodiment.

FIGS. 22A and 22B are views showing an example of how to use the insertion tool shown in FIGS. 21A to 21C.

FIGS. 23A and 23B are views showing the constitution of an insertion tool according to Modification example 1 and show an example of how to use the insertion tool.

FIGS. 24A to 24C are views showing the constitution of an insertion tool according to Modification example 2 and show an example of how to use the insertion tool.

FIGS. 25A to 25C are views showing the constitution of an insertion tool according to Modification example 3 and show an example of how to use the insertion tool.

FIG. 26A is a cross-sectional view showing a state in which the insertion step is performed, and FIG. 26B is a cross-sectional view showing a state in which the assisting means is disposed in the urethra.

FIGS. 27A and 27B are views showing insertion tools according to Modification examples 4 and 5.

FIGS. 28A to 28C are views showing insertion tools according to Modification examples 6 to 8.

FIG. 29A is a cross-sectional view showing a state in which the maintenance step has been performed by using predetermined maintenance means, and FIG. 29B is a cross-sectional view showing a state in which the maintenance step has been performed by using the maintenance means according to Modification example 1.

FIG. 30A is a cross-sectional view of the urethra having undergone the treatment step, and FIG. 30B is a view showing an example of how to use maintenance means according to Modification example 2.

FIG. 31 is a view showing an example of how to use maintenance means according to Modification example 3.

FIGS. 32A and 32B are views showing an example of how to use maintenance means according to Modification example 4.

FIG. 33 is a view showing a modification example of a supporting tool shown in FIG. 30B.

FIGS. 34A and 34B are views showing an example of how to use maintenance means according to Modification examples 5 and 6.

FIGS. 35A and 35B are views showing an example of how to use maintenance means according to Modification examples 7 and 8.

FIG. 36 is a view for illustrating the constitution of maintenance means according to Modification example 9.

FIGS. 37A and 37B are views showing an example of how to use maintenance means according to Modification example 10.

FIG. 38 is a cross-sectional view showing an example of how to use the maintenance means according to Modification example 10.

### DETAILED DESCRIPTION

Hereinafter, embodiments of the present invention will be described with reference to drawings. In the following description, the dimensional ratios of the drawings are magnified for convenience and differ from the actual ratios in some cases.

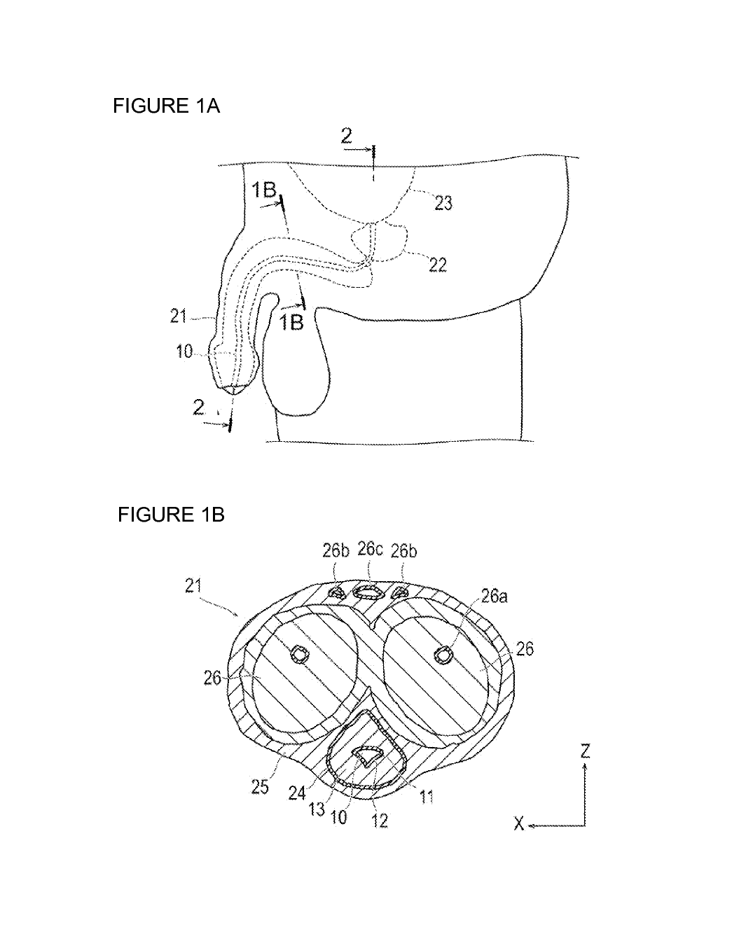

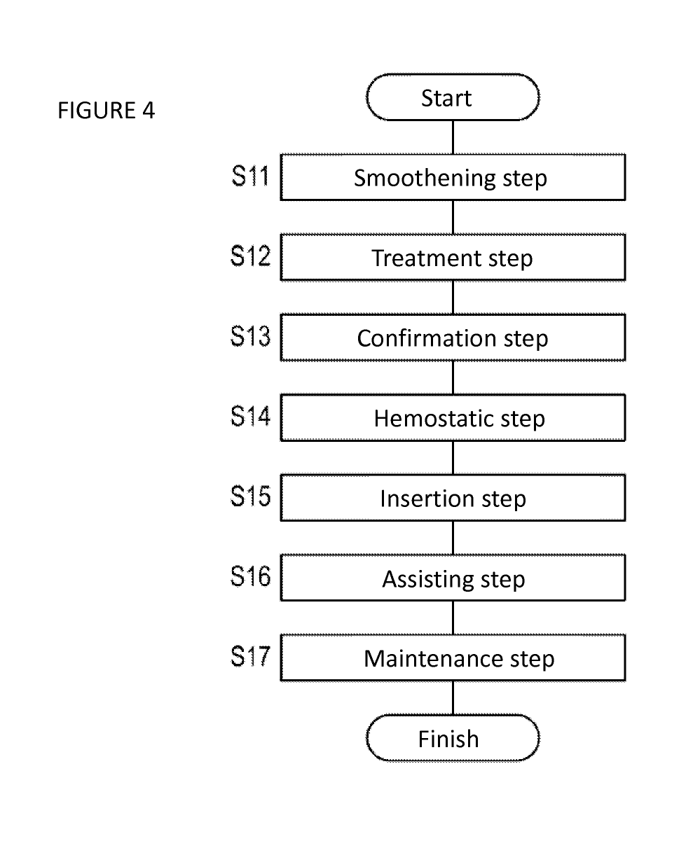

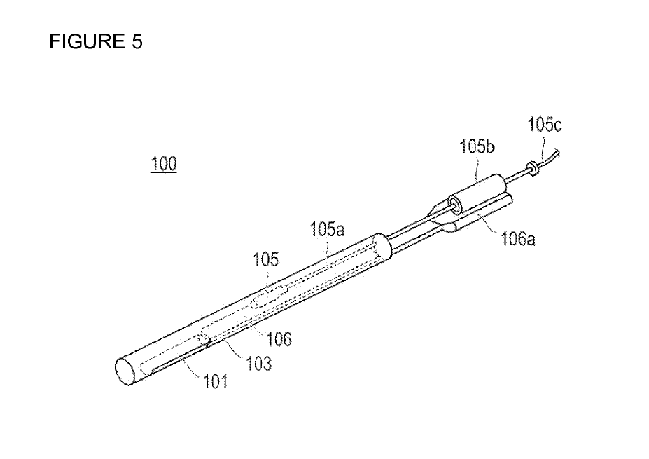

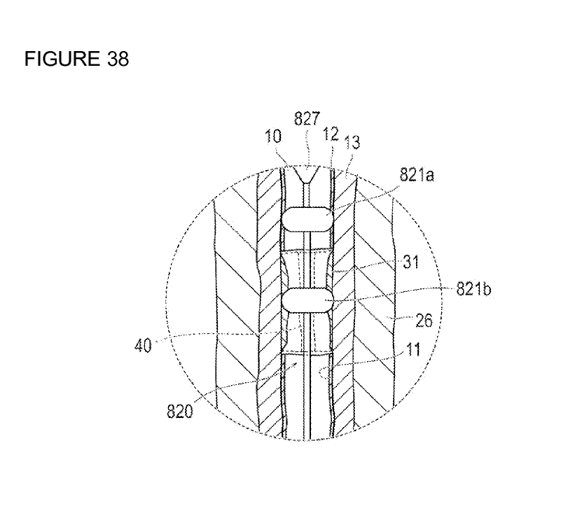

FIGS. 1A to 3B are views schematically showing the urethra of a biological body suffering from urethral stricture, and FIG. 4 is a view showing the entire steps of the treatment method according to the present exemplary embodiment. FIGS. 5 to 38 are views for illustrating medical instruments or the respective steps used in the treatment method according to the present exemplary embodiment.

The present invention described based on embodiments relates to a urethral stricture treatment method, and particularly relates to a treatment method inhibiting restenosis (reocculusion) from occurring after a stenosed portion formed in the urethra undergoes a procedure. First, the structure of periphery of the urethra of a biological body, urethral stricture, mechanism of the development of restenosis, and the like will be described.

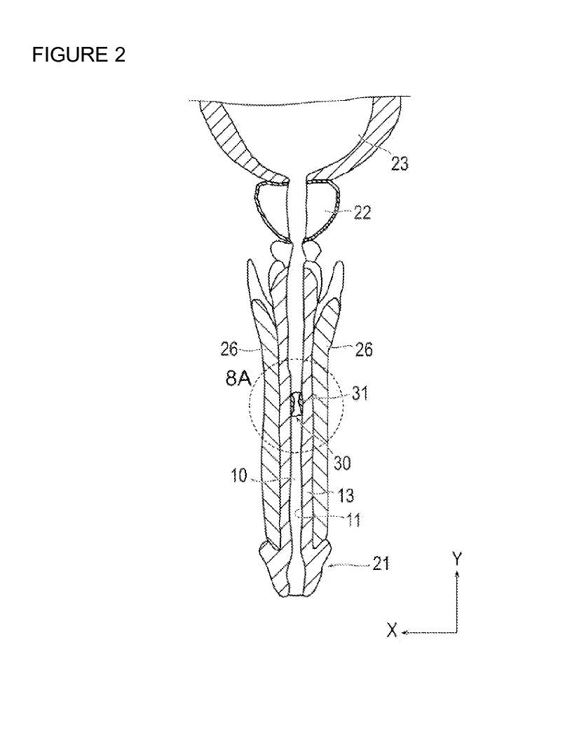

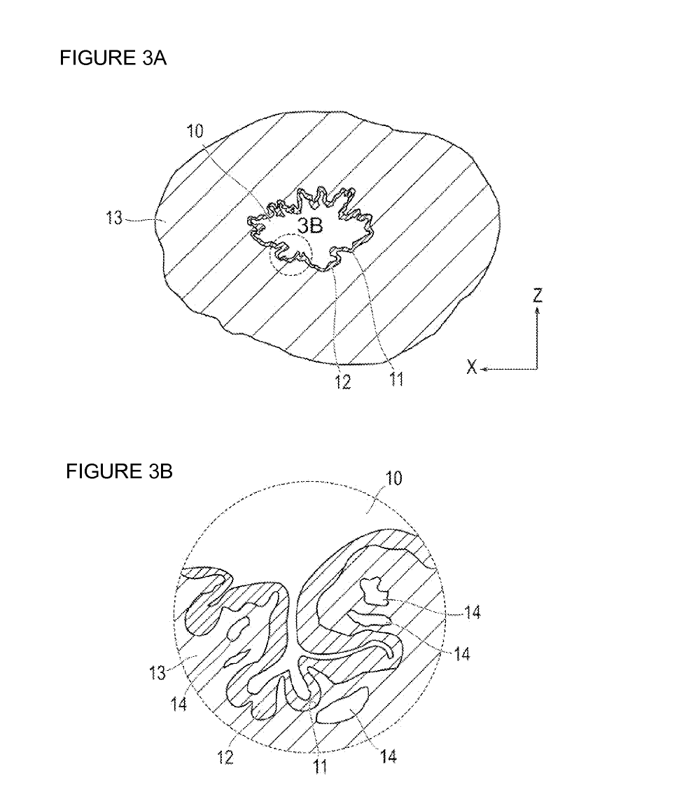

FIGS. 1A to 3B schematically show a male urethra 10 and the periphery of the male urethra 10. As shown in FIG. 1A, the urethra 10 passes through the inside of a penis 21 and the inside of a prostate 22 and leads to a bladder 23 in the lower abdomen of the biological body. As shown in FIGS. 1B and 3A, an inner surface 11 of the urethra 10 is covered with a urethral mucosa 12. A corpus spongiosum penis 13 is on the outside of the urethral mucosa 12. As shown in FIG. 3B, in the corpus spongiosum penis 13, there are blood vessels 14 called caverns of corpus spongiosum running in the form of network. In each of the drawings, an X-axis indicates a width direction (horizontal direction of FIG. 1B) of the urethra, a Y-axis indicates a direction (vertical direction of FIG. 2) in which the urethra extends, and a Z-axis indicates a height direction (vertical direction of FIG. 1B) of the urethra.

As shown in FIG. 1B, the corpus spongiosum penis 13 is covered with a tunica albuginea 24, and a fascia 25 and a corpus cavernosum penis 26 are on the outside of the tunica albuginea 24. An arteria profunda penis 26a is in the corpus cavernosum penis 26, and an arteria dorsalis penis 26b and a venae dorsalis penis 26c are above the corpus cavernosum penis 26.

Urethral stricture is a disease that can be caused by the following mechanism. In the process in which the urethral mucosa 12 or the corpus spongiosum penis 13 is damaged by, for example, trauma or inflammation and then healed, the tissue of the urethral mucosa 12 or the tissue of the corpus spongiosum penis 13 surrounding the urethral mucosa 12 undergoes fibrosis or cicatrization. Consequentially, a scar tissue 31 can be formed in the inner surface 11 of the urethra as shown in FIG. 2, and as a result, the lumen of the urethra 10 is narrowed. In the urethra 10, the cross-sectional area of the site (stenosed site 30) at which the scar tissue 31 has been formed becomes narrower than that of other sites of the urethra 10. Accordingly, urine does not easily pass through the site. The urethral stricture causes micturition disorders that can hinder smooth urination or can cause complete urinary retention.

As a urethral stricture treatment method, for example, a method has been tried in which a medical instrument such as a bougie (urethral dilator), a balloon, a cold knife, or a laser is transurethrally inserted into the urethra so as to treat the stenosed portion by dilating the stenosed portion or making an incision in the stenosed portion by using the medical instrument. Although the treatment method can bring about a temporary effect, the stenosed site 30 can be formed again after the treatment is provided, and thus urethral stricture can be highly likely to reoccur. Because the scar tissue 31 has liquid permeability, the periphery of the scar tissue 31 or the corpus spongiosum penis 13 under the scar tissue 31 is constantly exposed to liquid (for example, urine or blood) flowing in the urethra 10. Consequentially, an inflammatory reaction is continuously induced, reconstruction of epithelial cells is hindered, and as a result, urethral stricture can reoccur.

The treatment method according to the present embodiment is a treatment method that can help prevent the recurrence of urethral stricture by inhibiting reconstruction properties of an epithelial function (reconstruction properties of the urethral mucosa 12) from deteriorating due to the influence of the liquid flowing in the urethra 10. As shown in FIG. 4, the treatment method can include a smoothening step S11 of smoothening the inner surface of the urethra, a treatment step S12 of performing a predetermined treatment on scar tissue so as to form a treatment portion, a confirmation step S13 of confirming whether or not the treatment portion has been formed, a hemostatic step S14 of stopping hemorrhage when hemorrhage occurs in the urethra, an assisting step S16 of imparting an epithelial function to the treatment portion, and a maintenance step S17 of maintaining a state in which assisting means used in the assisting step is in contact with the treatment portion.

The treatment step S12 will be described.

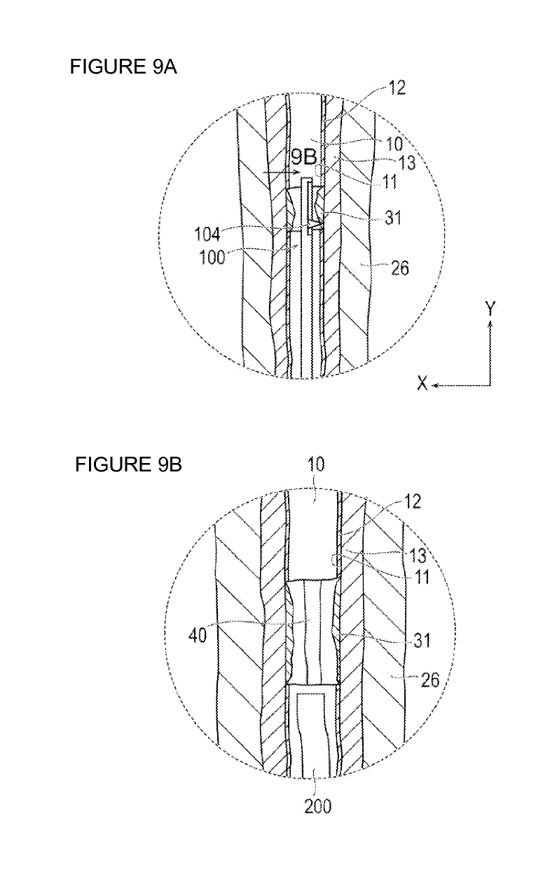

In accordance with an exemplary embodiment, the treatment step S12 is a step of treating the scar tissue 31 by using a treatment tool 100 transurethrally inserted into the urethra 10 so as to form a treatment portion 40 (see FIGS. 9A and 9B) to which an epithelial function can be imparted. Herein, the treatment can include, for example, incision, cutting, excision, and the like of the scar tissue 31.

As described above, when the corpus spongiosum penis 13 under the scar tissue 31 is exposed to liquid such as urine, an inflammatory reaction can occur. Due to urination, urine irregularly and repeatedly moves in the urethra 10. Therefore, through the scar tissue 31, the corpus spongiosum penis 13 easily and frequently comes into contact with the urine. Furthermore, even if an attempt is made to protect the corpus spongiosum penis 13 from liquid, because the scar tissue 31 is on the corpus spongiosum penis 13, it can be difficult to directly perform medical treatment by a transurethral approach. In the treatment step S12, the site of the urethra 10 at which the scar tissue 31 has been formed is treated so as to form a supply route that enables the supply of epithelial engraftment factors from the corpus spongiosum penis 13 under the scar tissue 31. As described later, by imparting an epithelial function to the treatment portion 40 formed in the treatment step S12, the reconstruction of the epithelial function can be assisted by using the treatment portion 40 as a foundation.

In the present specification, the “epithelial function” refers to a barrier function of preventing or reducing the likelihood of contact between the corpus spongiosum penis 13 and liquid such as urine as well as inflammatory components contained in the urethra.

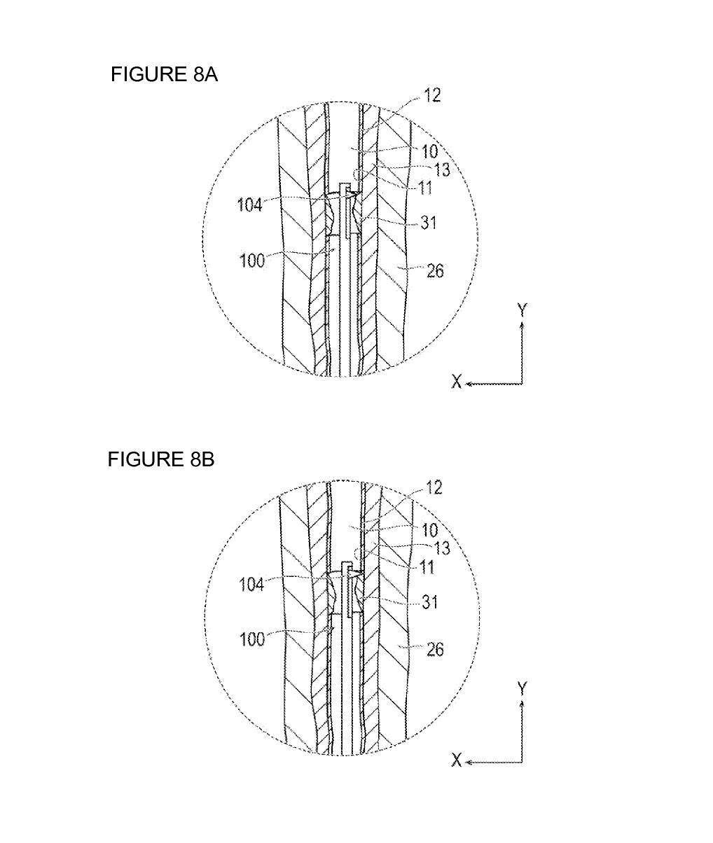

The treatment step S12 can include, for example, a step of making an incision in the scar tissue 31. As described later with reference, for example, to FIGS. 8A-9B, when an incision is made in the scar tissue 31 formed in the urethra 10, the corpus spongiosum penis 13 under the scar tissue 31 is exposed. The site exposed from the scar tissue 31 becomes the treatment portion 40. In the assisting step S16 that will be described later, the epithelial function may be imparted only to the site exposed from the scar tissue 31 or may be imparted to a site including the exposed site and the periphery thereof. If the epithelial function is imparted to a wider range of area, the treatment effect for preventing the recurrence of urethral stricture can be improved.

Next, an example of the treatment tool 100 used in the treatment step S12 will be described with reference to FIGS. 5 to 7B.

The treatment tool 100 can include a sheath 103 in which a window portion 101 is formed, a dilative deformation portion 105 including a blade portion 104, and a slide portion 106 used for opening or closing the window portion 101 of the sheath 103.

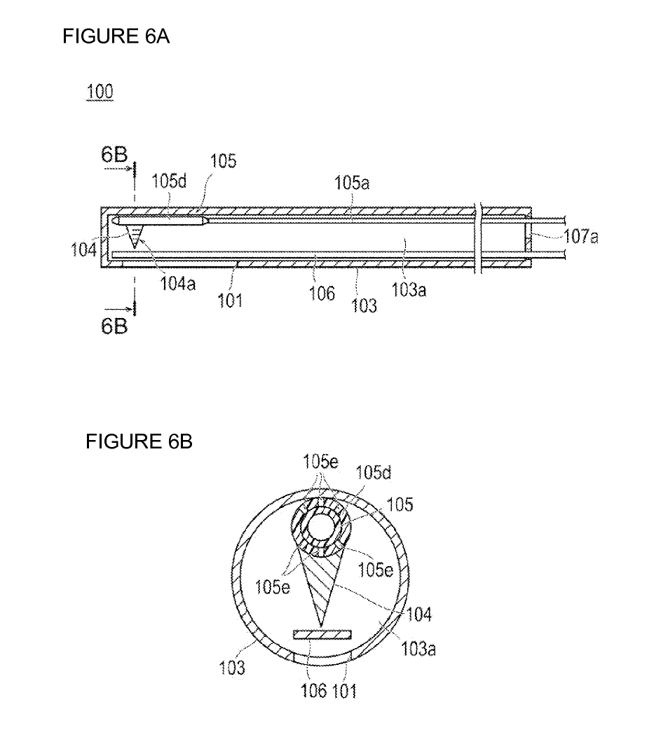

As shown in FIG. 6A, the sheath 103 can include a lumen 103a extending to the proximal end of the sheath 103 from the distal end of the sheath 103. In the lumen 103a of the sheath 103, the dilative deformation portion 105 and the slide portion 106 can be accommodated in a state of being able to freely move back and forth in the axial direction. The window portion 101 formed in the sheath 103 is constituted with a through hole having a predetermined length that is formed along the axial direction.

The material, the external shape, and the like of the sheath 103 is not particularly limited, as long as the sheath 103 can be inserted into a biological body. Furthermore, the size of the window portion 101 formed in the sheath 103, the position of the window portion 101, are not particularly limited, as long as the distal end portion of the blade portion 104 can protrude outside the sheath 103 when the dilative deformation portion 105 is dilated as described later.

The dilative deformation portion 105 is constituted with a balloon that can be deformed by dilation and contraction. As shown in FIG. 5, the proximal end portion of a shaft 105a mounted on the dilative deformation portion 105 is disposed on the outside of the sheath 103. Moreover, the shaft 105a can include a port 105b that can be connected to a fluid tube 105c used for supplying a pressure medium injected into the dilative deformation portion 105 for dilating the dilative deformation portion 105. The pressure medium can be supplied into and discharged from the dilative deformation portion 105 by known medium supply means, for example, such as a syringe pump which is not shown in the drawing.

As shown in FIG. 6B, a hollow tube 105d equipped with the blade portion 104 is disposed on the outer circumferential area of the dilative deformation portion 105. The tube 105d is constituted such that it is deformed by dilation or contraction as the dilative deformation portion 105 is deformed by dilation or contraction. The tube 105d can include a plurality of slits 105e such that the tube 105d is smoothly deformed by dilation or contraction as the dilative deformation portion 105 is deformed. The tube 105d can be constituted with, for example, a resin material that can undergo elastic deformation.

The blade portion 104 disposed in the dilative deformation portion 105 can include a plurality of calibrations 104a that are arranged in a state of being separated from each other in the length direction (vertical direction of FIG. 6A) of the blade portion 104. The calibrations 104a can be used for confirming how deeply the blade portion 104 has been inserted into the scar tissue 31 when an incision is made in the scar tissue 31 by using the treatment tool 100 (see FIG. 8B). In accordance with an exemplary embodiment, the calibrations 104a are preferably colored such that they can be clearly seen in an image obtained by an imaging apparatus such as an endoscope. The calibrations 104a are preferably arranged such that they are separated from each other at an interval of, for example, 0.1 mm to 1 mm. Furthermore, numbers, signs, and the like for showing the position of the respective calibrations 104a can be marked on the blade portion 104. Moreover, the calibrations 104a can be constituted with concavities, convexities, and the like formed on the outer surface of the blade portion 104.

The material constituting the blade portion 104 is not particularly limited as long as the blade portion 104 can make an incision in the scar tissue 31. For example, the blade portion 104 can be constituted with a metal, hard plastic, or the like.

As shown in FIG. 5, a hand-side operation portion 106a, which can help control the movement of the slide portion 106 at the outside of the sheath 103, is disposed in the proximal end portion of the slide portion 106. In accordance with an exemplary embodiment, the hand-side operation portion 106a has a flat plate-like external shape such that it can be gripped by fingers. Herein, the external shape, the dimensions, the constituent material, and the like of the slide portion 106 and the hand-side operation portion 106a are not particularly limited, as long as they have a function of opening or closing the window portion 101 of the sheath 103.

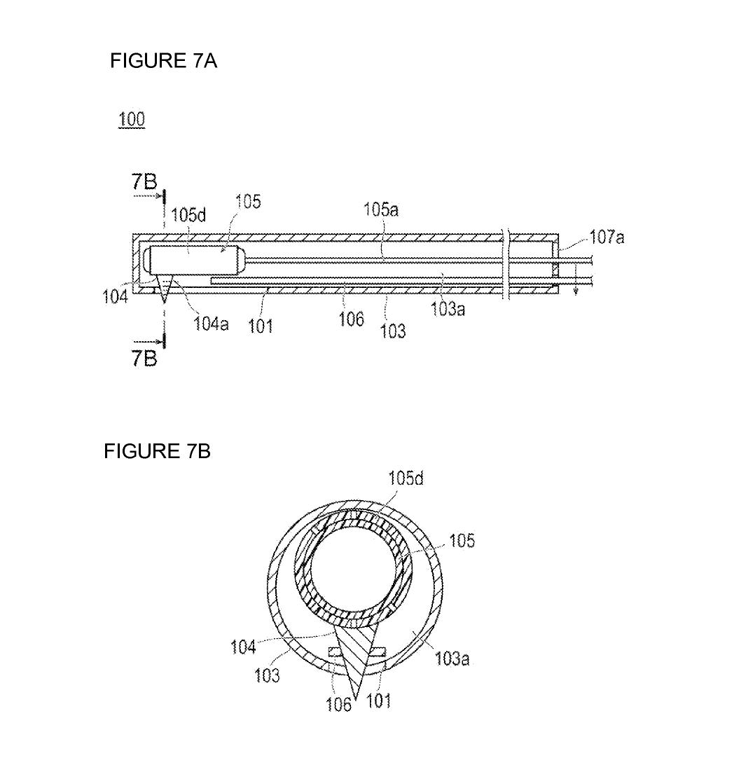

In accordance with an exemplary embodiment, the slide portion 106 included in the treatment tool 100 can have a function of protecting the inner surface 11 of the urethra from the blade portion 104 when the treatment tool 100 is inserted into the urethra 10. For example, as shown in FIGS. 6A and 6B, in a state in which the dilative deformation portion 105 has not yet been deformed by dilation, the blade portion 104 is accommodated in the sheath 103, and the window portion 101 of the sheath 103 is covered with the slide portion 106. In this way, the blade portion 104 can be stored in the sheath 103, and the safety of the blade portion 104 at the time of use can be improved. For making an incision in the scar tissue 31 by using the blade portion 104, the slide portion 106 is moved to a position so as to open the window portion 101 as shown in FIG. 7A. If the slide portion 106 is located in such a position, and the dilative deformation portion 105 is dilated, the blade portion 104 can protrude from the window portion 101.

As shown in FIG. 7A, the shaft 105a mounted on the dilative deformation portion 105 can be led to the outside of the sheath 103 through an opening portion 107a formed in the proximal end portion of the sheath 103. The dilative deformation portion 105 is supported by the shaft 105a such that it can swing in the opening portion 107a. Therefore, when the dilative deformation portion 105 is deformed by dilation, due to the reactive force generated when the dilative deformation portion 105 is pushed against the inner surface of the sheath 103, the shaft 105a moves in the opening portion 107a in the direction of the arrow shown in the drawing. As a result, the dilative deformation portion 105 moves to the lower side in the drawing, and the blade portion 104 mounted on the dilative deformation portion 105 is guided to the window portion 101.

The dilative deformation portion 105 is constituted with a balloon as described above. Accordingly, if the pressure of the pressure medium injected into the dilative deformation portion 105 is adjusted, the degree of deformation of the dilative deformation portion 105 in the radial direction can be adjusted. The blade portion 104 is mounted on the dilative deformation portion 105. Accordingly, when the scar tissue 31 is treated by using the treatment tool 100, if an operation of stepwise deforming the dilative deformation portion 105 is performed, unnecessary damage to the scar tissue 31 and the corpus spongiosum penis 13 by the blade portion 104 can be prevented.

Next, the procedure for forming the treatment portion 40 by using the treatment tool 100 will be described with reference to FIGS. 8A, 8B, and 9A. Herein, the drawings referred to are enlarged views of the portion corresponding to a portion 8A indicated by the dotted line in FIG. 2.

As shown in FIG. 8A, the treatment tool 100 is inserted into the urethra 10. At the stage of inserting the treatment tool 100 into the urethra 10, the window portion 101 of the sheath 103 is in a state of being closed by the slide portion 106. At the time of inserting the treatment tool 100, a known imaging apparatus such as an endoscope can be used. In this case, the treatment tool 100 can be inserted into the urethra 10 through a channel included in the imaging apparatus. Furthermore, the treatment tool 100 can be inserted into the urethra 10 by using, for example, a guide sheath known in the medical field.

Thereafter, the window portion 101 of the sheath 103 is caused to face the scar tissue 31. Then the dilative deformation portion 105 included in the treatment tool 100 is deformed by dilation such that scar tissue 31 is punctured by the blade portion 104 as shown in FIG. 8B. At this time, the treatment for the scar tissue 31 is performed while avoiding the direction in which the corpus cavernosum penis 26 is positioned in the circumferential direction of the urethra 10. Although the corpus cavernosum penis 26 is in a diagonally upper position of the urethra 10 as shown in FIG. 1B, if the blade portion 104 reaches and damages the corpus cavernosum penis 26, this may lead to a problem of impairment of erectility of the penis 21. Therefore, if the treatment is performed in a direction in which the corpus cavernosum penis 26 is not positioned, specifically, in a direction outside a range of a predetermined angle (for example, −20° to −60° and 20° to 60°) in a diagonally upper position from the center of the urethra 10, the treatment step S12 can be performed without damaging the corpus cavernosum penis 26. When the treatment is performed on the urethra in the vicinity of the prostate 22, in order to more reliably prevent the damage of the corpus cavernosum penis 26, it is preferable to perform the treatment downward in an approximately vertical direction, for example, a direction of 120° to 240°, and more preferably a direction of 150° to 210°.

For example, the treatment performed by the blade portion 104 is administered plural times while changing the depth stepwise in the thickness direction of the scar tissue 31. If the treatment is performed while changing the depth stepwise, unnecessarily damaged to the corpus spongiosum penis 13 by the blade portion 104 can be prevented. As described above, if the degree of dilative deformation of the dilative deformation portion 105, which is included in the treatment tool 100, is adjusted, the length of the blade portion 104 protruding from the window portion 101 of the sheath 103 can be adjusted. Accordingly, the scar tissue 31 with the blade portion 104 can be stuck to a depth changed stepwise. Herein, the degree (depth) of a puncture made in the scar tissue 31 by the blade portion 104 at a time and the final depth of the puncture made by the blade portion 104 can be optionally set according to the thickness and the like of the scar tissue 31. In the present exemplary embodiment, the scar tissue 31 can be treated to the depth almost reaching the corpus spongiosum penis 13. However, the treatment may be performed to a depth enough to reach the corpus spongiosum penis 13.

While the treatment step S12 is being performed, or alternatively, after the treatment step S12 is performed, for example, a depth diagnosing step of diagnosing the depth of the treatment performed on the scar tissue 31 (the depth at which the blade portion 104 is stuck into the scar tissue 31) can be performed. If this step is performed, the depth of the scar tissue that has been treated can be confirmed. Consequentially, unnecessary damage to the corpus spongiosum penis 13 to by the blade portion 104 can be prevented. For example, the depth can be diagnosed by confirming the calibrations 104a arranged in the blade portion 104 by using an imaging apparatus or the like. Moreover, the depth can be diagnosed by using other methods such as a method of using a guide wire for medical use that can include calibrations, a measurement instrument that can be inserted into the urethra, or the like. Furthermore, when pressure has been applied to the inner wall of the urethra 10 due to hydraulic pressure resulting from a cystoscope, by observing an incision layer which spreads (is cut) along the circumference by using the cystoscope, a determination can be made whether the treatment has been performed to a sufficient degree.

Subsequently, as shown in FIG. 9A, the blade portion 104 of the treatment tool 100 is moved in the direction in which the scar tissue 31 extends (vertical direction in the drawing) so as to make an incision in the scar tissue 31. By performing the operation, the corpus spongiosum penis 13 from the scar tissue 31 can be exposed. In order to expose the corpus spongiosum penis 13 over a broader range, for example, the blade portion 104 may be rotated in the circumferential direction of the urethra 10, or alternatively, an operation of moving the blade portion 104 in the direction in which the scar tissue 31 extends may be repeated plural times.

According to the progress of the treatment step S12, the confirmation step S13 is performed.

The confirmation step S13 is a step of confirming whether or not the treatment portion 40 has been formed by using a diagnostic tool 200 transurethrally inserted into the urethra 10 as shown in FIG. 9B. Herein, FIG. 9B is an enlarged cross-sectional view of the urethra 10 seen from the direction indicated by arrow 9B shown in FIG. 9A.

As the diagnostic tool 200, for example, an imaging apparatus (known imaging apparatus for medical use such as an endoscope) can be transurethrally inserted into the urethra 10 and can capture images of the inside of the urethra 10. If an imaging apparatus is used as the diagnostic tool 200, the confirmation step S13 can include a step of determining whether or not at least a portion of the corpus spongiosum penis 13 has been exposed from the scar tissue 31 based on an diagnostic image captured by the imaging apparatus. The treatment step S12 described above is performed until the corpus spongiosum penis 13 is confirmed by the confirmation step S13. Herein, the confirmation step S13 can be performed by using an apparatus (for example, an ultrasonic diagnostic apparatus) other than the imaging apparatus such as an endoscope, an X-ray imaging apparatus using a contrast agent, and the like.

The confirmation step S13 can include, for example, a step of visually determining whether or not the corpus spongiosum penis 13 has been exposed based on the color change of the diagnostic image captured by the diagnostic tool 200.

When an incision is made in the scar tissue 31, and thus the thickness of the scar tissue 31 is reduced in the treatment step S12 as described above, due to the influence of blood flowing in the corpus spongiosum penis 13, the thin scar tissue 31 appears red or reddish in the image. By the simple operation of visually confirming the color of the scar tissue 31, the depth to the corpus spongiosum penis 13 can be determined. If hemorrhage occurs in the site having undergone treatment, whether or not the corpus spongiosum penis 13 has been exposed may be determined by visually confirming the hemorrhage.

After the exposure of the corpus spongiosum penis 13 is confirmed by the confirmation step S13, the treatment step S12 ends. The corpus spongiosum penis 13 exposed from the scar tissue 31 constitutes the treatment portion 40 to which the epithelial function can be imparted as described above.

Next, modification examples of the treatment tool used in the treatment step S12 will be described.

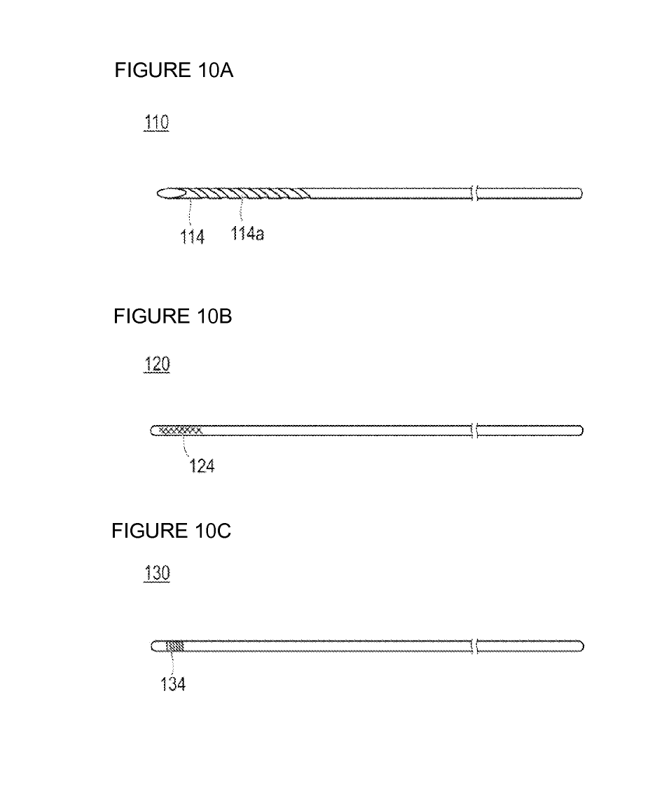

FIG. 10A shows a treatment tool 110 according to a Modification example 1.

The treatment tool 110 can include a needle-like blade portion 114 in which spiral grooves 114a are formed. If the blade portion 114 is stuck into the scar tissue 31 from the distal end side of the blade portion 114 and is caused to reciprocate in the direction in which the scar tissue 31 extends, the scar tissue 31 can be scraped off. When hemorrhage occurs in the treatment step S12, blood can be discharged through the spiral grooves 114a formed in the blade portion 114. Herein, the inside of the blade portion 114 may be processed such that it becomes a hollow portion having a lumen. If the lumen is formed, blood can be discharged to the outside of the urethra 10 through the lumen.

FIG. 10B shows a treatment tool 120 according to Modification example 2.

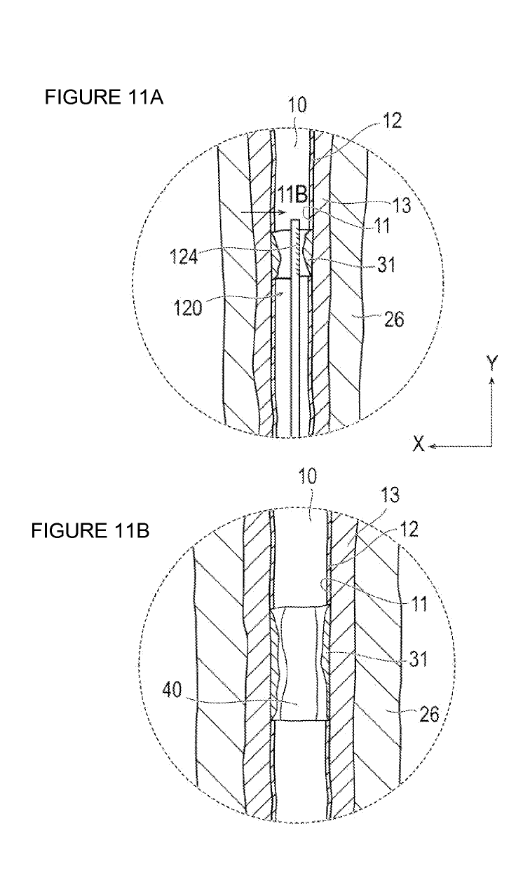

The treatment tool 120 can include a polishing portion 124 that is constituted such that it can scrape the scar tissue 31. As shown in FIG. 11A, in a state in which the polishing portion 124 comes into contact with the scar tissue 31, if the polishing portion 124 is caused to reciprocate in the direction (direction indicated by an arrow in the drawing) in which the scar tissue 31 extends, the scar tissue 31 can be scraped off.

As shown in FIG. 11B, if the scar tissue 31 is scraped off, the corpus spongiosum penis 13 is exposed. FIG. 11B is an enlarged cross-sectional view of the urethra 10 seen from the direction of arrow 11B shown in FIG. 11A. The aforementioned treatment of making an incision in the scar tissue 31 and the treatment of scraping the scar tissue 31 can be simultaneously or alternately performed in a single treatment step S12.

FIG. 10C shows a treatment tool 130 according to Modification example 3.

The treatment tool 130 can include a grater-like bump portion 134 constituted such that it can scrape the scar tissue 31. In the state in which the bump portion 134 comes into contact with the scar tissue 31, if the bump portion 134 is caused to reciprocate in the direction in which the scar tissue 31 extends, the corpus spongiosum penis 13 can be exposed by squeezing the scar tissue 31.

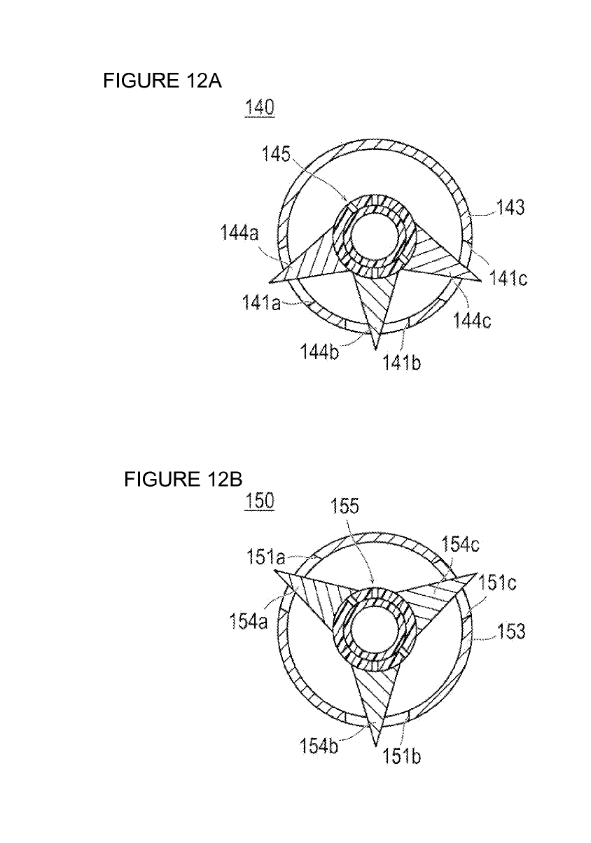

FIG. 12A shows a treatment tool 140 according to Modification example 4.

Although the treatment tool 100 shown in FIG. 5 is constituted with a single blade portion 104, the treatment tool 100 can include a plurality of blade portions. For example, as shown in FIG. 12A, the treatment tool 100 can include three blade portions 144a to 144c. If the blade portions 144a to 144c are arranged such that they protrude downwardly as shown in the drawing, the corpus cavernosum penis 26 can be suitably prevented from being damaged in the treatment step S12. Due to the dilation of a dilative deformation portion 45, three of the blade portions 144a to 144c can simultaneously protrude. Accordingly, the treatment step S12 can be effectively performed. In accordance with an exemplary embodiment, a sheath 143 can include three window portions 141a to 141c equal to the blade portion in number. The treatment tool can include three or more blade portions.

FIG. 12B shows a treatment tool 150 according to Modification example 5.

As shown in the present modification example, blade portions 154a to 154c may be arranged at an equal interval in the circumferential direction of a dilative deformation portion 155. If the blade portions are arranged as described above, the scar tissue 31 can be effectively treated. Moreover, in this modification example, the treatment tool can include three or more blade portions. If the number of the blade portions is increased, even though a shallow incision is made in each incision site, the treatment portion 40 can be formed.

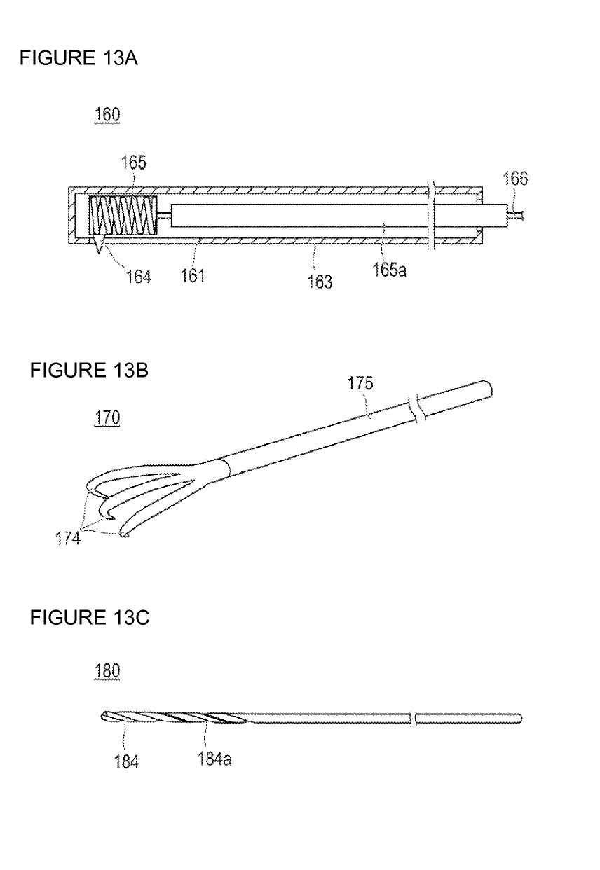

FIG. 13A shows a treatment tool 160 according to Modification example 6.

The treatment tool 160 can include a coil 165 that can be deformed by dilation, a blade portion 164 mounted on the coil 165, a shaft 165a mounted on the proximal end portion of the coil 165, and a sheath 163 in which a window portion 161 can be formed.

In the treatment tool 100 shown in FIG. 5, the dilative deformation portion 105 can be constituted with a balloon. In contrast, the dilative deformation portion 165 of the treatment tool 160 according to the present modification example is constituted with the coil 165 that can dilate and contract. For example, the coil 165 can be constituted such that it is deformed by dilation and contraction in tandem with a pushing-pulling operation or a rotation operation of a predetermined tugging member 166 connected to the coil 165. Furthermore, for example, the coil 165 may be constituted such that it is deformed by being covered with another sheath or the like. Moreover, the treatment tool may be constituted with a coil having a plurality of sharp bumps or blades on the outer surface of the coil. By performing an operation of contracting-dilating the coil, the treatment tool can be smoothly inserted into the urethra 10 and the treatment in the urethra 10 can be smoothly performed. In addition, the coil may have a structure that plays the role of a blade.

FIG. 13B shows a treatment tool 170 according to Modification example 7.

The treatment tool 170 can include claw portions 174 that are constituted to be able to scrape the scar tissue 31 and a hand-side operation portion 175 that is disposed at the proximal end side of the claw portions 174. The distal end portion of each of the claw portions 174 can have a curved shape such that the claw portions 174 can hook and scrape the scar tissue 31. In a state in which the claw portions 174 come into contact with the scar tissue 31, if the claw portions 174 are caused to reciprocate in the direction in which the scar tissue 31 extends, the scar tissue 31 can be scraped and the corpus spongiosum penis 13 can be exposed. The number of the claw portions 174 provided in the treatment tool is not limited to three and can be appropriately changed.

FIG. 13C shows a treatment tool 180 according to Modification example 8.

The treatment tool 180 can include a drill-like blade portion 184 in which spiral grooves 184a are formed. If the blade portion 184 is stuck into the scar tissue 31 from the distal end portion of the blade portion 184 and caused to reciprocate in the direction in which the scar tissue 31 extends, the scar tissue 31 can be scraped off.

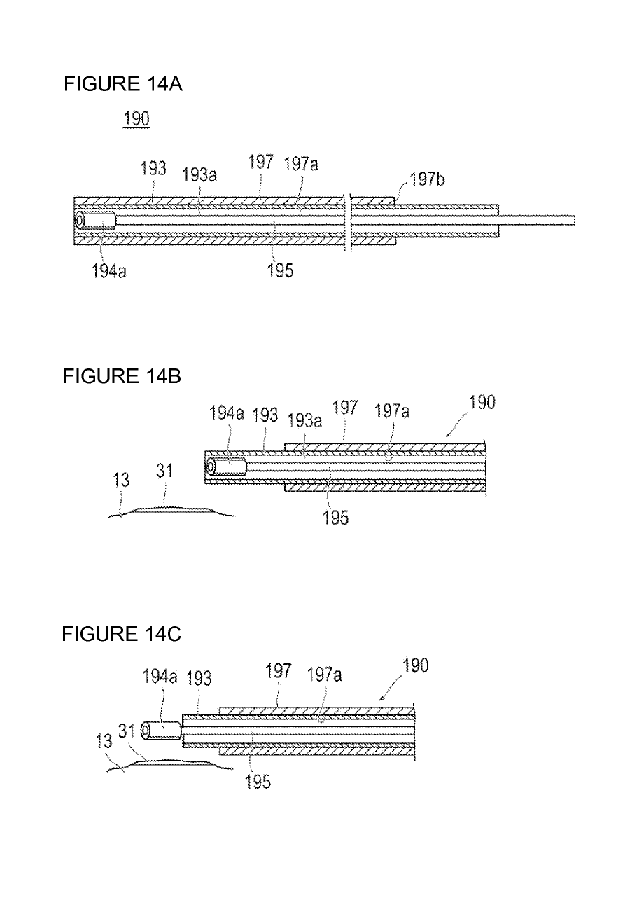

FIGS. 14A-C and 15A-C show a treatment tool 190 according to Modification example 9.

The treatment tool 190 can include a plurality of blade portions 194a, 194b, and 194c that are constituted to be able to be deformed by dilation, a shaft 195 on which the respective blade portions 194a, 194b, and 194c are mounted, a first sheath 193 in which a lumen 193a, which accommodates the respective blade portions 194a, 194b, and 194c and allows the blade portions to move back and forth, is formed, and a second sheath 197 in which a lumen 197a, which accommodates the first sheath 193 accommodating the respective blade portions 194a, 194b, and 194c and allows the first sheath 193 to move back and forth, is formed.

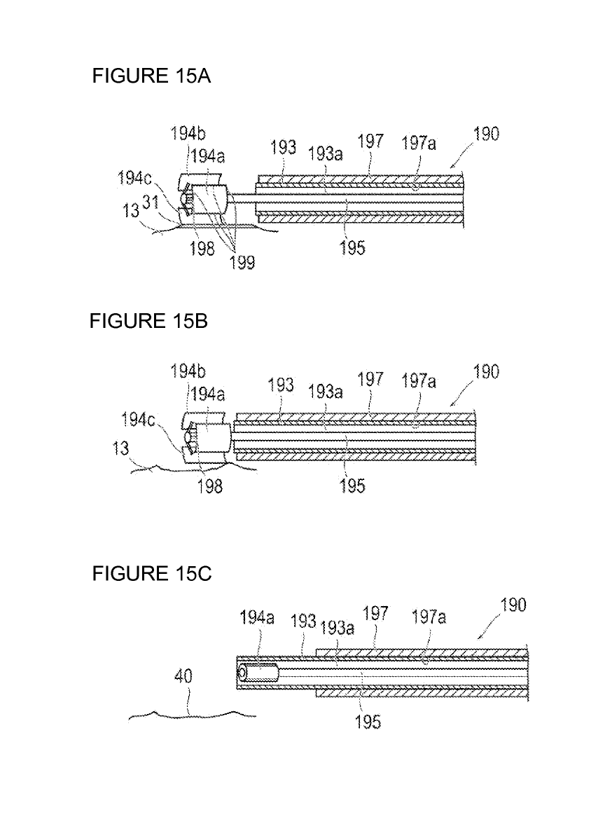

As shown in FIG. 15A, the respective blade portions 194a to 194c are mounted on the shaft 195 through an arm portion 198 that is constituted to be able to be folded toward the central axis side of the shaft 195. As shown in FIGS. 14A and 14B, when the respective blade portions 194a to 194c are accommodated in the lumen 193a of the first sheath 193, the arm portion 198 is folded, and the respective blade portions 194a to 194c are arranged such that they become close to each other in the central position in the radial direction of the shaft 195. As a result, the respective blade portions 194a to 194c are accommodated in the lumen 193a of the first sheath 193, in a state of being miniaturized compact portions. As shown in FIG. 15A, when the respective blade portions 194a to 194c protrude from the lumen 193a of the first sheath 193, the folded arm portion 198 is opened, and the respective blade portions 194a to 194c are deformed by dilation.

For example, the arm portion 198 can be constituted with a metal material or a resin material processed so as to be able to undergo elastic deformation, such that the respective blade portions 194a to 194c can be smoothly accommodated in the lumen 193a of the first sheath 193, and that the arm portion 198 can be smoothly opened in tandem with the protrusion of the respective blade portions 194a to 194c from the lumen 193a of the first sheath 193.

The respective blade portions 194a to 194c are constituted such that the external shape of the respective blade portions 194a to 194c become cylindrical when they are accommodated in the lumen 193a of the first sheath 193. Furthermore, the outer circumferential edges (peripheries of right and left and top and bottom) of the respective blade portions 194a to 194c form a blade surface 199. Accordingly, if an operation of pushing and pulling the respective blade portions 194a to 194c in the axial direction of the first sheath 193 and an operation of rotating the respective blade portions 194a to 194c around the axis of the first sheath 193 are performed, incision and cutting of the scar tissue 31 can be performed.

As shown in FIG. 14A, both the proximal end portion of the first sheath 193 and the proximal end portion of the shaft 195 are led outside a lumen 197a of the second sheath 197 through an opening portion 197b formed in the proximal end portion of the second sheath 197. If each of the proximal end portion of the first sheath 193, the proximal end portion of the shaft 195, and the proximal end portion of the second sheath 197 is manually operated, each member can move back and forth or rotate.

An example of how to use the treatment tool 190 will be described with reference to FIGS. 14A-C and 15A-C.

As shown in FIG. 14A, for performing the treatment step S12, the respective blade portions 194a to 194c are accommodated in the lumen 193a of the first sheath 193, and the first sheath 193 is accommodated in the lumen 197a of the second sheath 197. In this state, the treatment tool 190 is inserted into the urethra 10.

As shown in FIG. 14B, the treatment tool 190 is guided to the vicinity of the scar tissue 31, and then the first sheath 193 accommodating the respective blade portions 194a to 194c is caused to protrude from the lumen 197a of the second sheath 197.

As shown in FIG. 14C, the distal end portion of the first sheath 193 is brought close to the scar tissue 31 and positioned in the scar tissue 31, and then the respective blade portions 194a to 194c are caused to protrude from the lumen 193a of the first sheath 193.

As shown in FIG. 15A, the respective blade portions 194a to 194c protruding from the lumen 193a of the first sheath 193 can be deformed by dilation.

As shown in FIG. 15B, if the respective blade portions 194a to 194c are pushed, pulled, or rotated, the treatment portion 40 can be formed by scarping the scar tissue 31. Moreover, when the respective blade portions 194a to 194c are caused to protrude from the lumen 193a of the first sheath 193, the blade surface 199 of the respective blade portions 194a to 194c can be inserted into the scar tissue 31. If the respective blade portions 194a to 194c are moved in the direction (horizontal direction in the drawing) in which the scar tissue 31 extends after the blade surface is stuck into the scar tissue, an incision can be made in the scar tissue 31 and to form the treatment portion 40. The degree of dilative deformation of the respective blade portions 194a to 194c can be adjusted according to the degree of elastic deformation of the arm portion 198. Therefore, if the degree of deformation is adjusted beforehand, the corpus spongiosum penis 13 can be prevented from being unnecessarily damaged by the respective blade portions 194a to 194c.

As shown in FIG. 15C, after the treatment portion 40 is formed, the respective blade portions 194a to 194c are accommodated in the lumen 193a of the first sheath 193. By an operation of pulling back the respective blade portions 194a to 194c to the proximal end side, the blade portions can be simply accommodated in the lumen 193a. At this time, along with the movement of the respective blade portions 194a to 194c, the scar tissue 31 collected by excision can be accommodated in the first sheath 193. In this way, scattering of the scar tissue 31 can be prevented.

Each of the treatment tools illustrated in the drawings is an example of an instrument usable in the treatment step S12, but the treatment step S12 can be performed by using other treatment tools. For example, various treatment tools can be used that can transurethrally treat the scar tissue 31 in a predetermined way, such as a known cold knife or laser scalpel used for incision, excision, or the like of tissues, a treatment tool having a structure in which a stent or the like is used as a dilative deformation portion including a blade portion, a treatment tool having a structure in which a blade portion included in a dilative deformation portion has a polishing function similar to that of a file, and a treatment tool constituted to scrape off a blade surface similarly to a plane. Moreover, the treatment step S12 may be performed by using each of the treatment tools singly or by using a plurality of any objects concurrently.

Next, the smoothening step S11 will be described.

The smoothening step S11 is performed for making it possible to smoothly perform the treatment step S12. As shown in FIGS. 3A and 3B, usually, the inner surface 11 of the urethra is in the form of a surface having irregular concavities and convexities. Moreover, because the thickness of the scar tissue 31 formed on the inner surface 11 of the urethra is not uniform, the corpus spongiosum penis 13 is likely to be treated unnecessarily deeply in the treatment step S12. If the inner surface 11 of the urethra and the scar tissue 31 is smoothened and made even by performing the smoothening step S11, the treatment depth can be easily ascertained and the corpus spongiosum penis 13 can be prevented from being treated unnecessarily deeply.

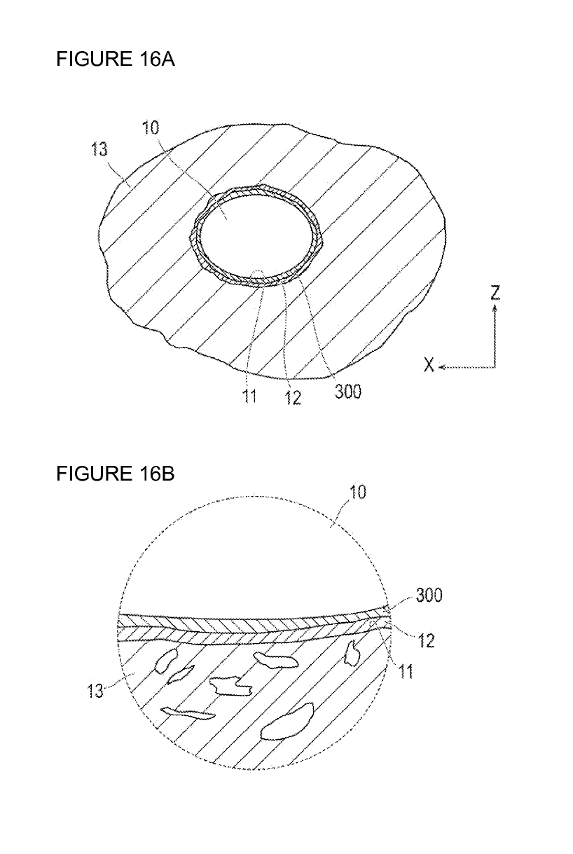

FIGS. 16A and 16B show a state in which the inner surface 11 of the urethra has been smoothened by using a predetermined smoothening tool 300.

The smoothening tool 300 is constituted with a stent that can be transurethrally inserted into the urethra 10. As the stent, for example, a known stent constituted to have self-dilating properties or a known stent constituted to be able to be deformed by dilation by a balloon catheter can be used. The outer diameter of the smoothening tool 300 is approximately the same as or greater than the inner diameter of the urethra 10.

As shown in FIG. 16A, when the smoothening tool 300 is disposed in the urethra 10, the inner surface 11 of the urethra is pressed and widens. As a result, as shown in FIG. 16B, the inner surface 11 of the urethra and the corpus spongiosum penis 13 in the vicinity of the inner surface 11 of the urethra are smoothened. Herein, FIG. 16B is an enlarged view of a portion of the inner surface 11 of the urethra shown in FIG. 16A.

If the treatment step S12 is performed in a state in which the inner surface 11 of the urethra has been smoothened, a treatment on the scar tissue 31 can be performed, such as making an incision at an appropriate depth. Herein, a predetermined opening portion or the like can be provided in the smoothening tool 300 such that the access to a site to be treated in the scar tissue 31 can be obtained.

Next, modification examples of the treatment tool used in the smoothening step S11 will be described. Each of the aforementioned smoothening tool 300 and smoothening tools 310 and 320 described below can be used singly. Moreover, as the smoothening tools, a plurality of any objects can be used concurrently.

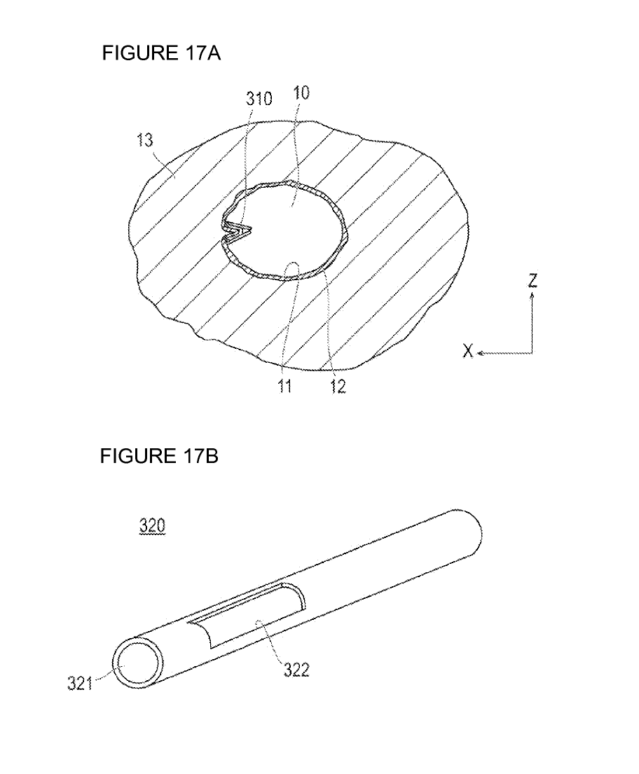

FIG. 17A shows an example of how to use a smoothening tool 310 according to Modification example 1.

The smoothening tool 310 can include a gripping member (a clip or the like) that can grip the inner surface 11 of the urethra. By gripping (pinching) a portion of the inner surface 11 of the urethra by using the smoothening tool 310 and applying tensile force to the entire inner surface 11 of the urethra, the inner surface 11 of the urethra is smoothened.

FIG. 17B shows a smoothening tool 320 according to Modification example 2.

The smoothening tool 320 can include a sheath that can be inserted into the urethra 10. The outer diameter of the smoothening tool 320 is approximately the same as or greater than the inner diameter of the urethra 10. When the smoothening tool 320 is inserted into the urethra 10, the inner surface 11 of the urethra is pressed and widens and is smoothened. In the smoothening tool 320, a lumen 321, which extends in the axial direction of the smoothening tool 320, and a window portion 322, through which the inside and outside of the lumen 321 communicate with each other, are formed. The smoothening tool 320 may be used as a guiding device by accommodating the treatment tool 100 or the like in the lumen 321 of the smoothening tool 320.

Each of the smoothening tools illustrated in the drawings is an example, and the inner surface 11 of the urethra can be smoothened by methods other than the methods using each of the illustrated smoothening tools. For example, a method of using a smoothening tool constituted with a spring-like coil, a method of smoothening the inner surface 11 of the urethra by using hydraulic pressure by supplying a fluid such as physiological salt solution into the urethra 10, a method of smoothening the inner surface 11 of the urethra by gripping the inner surface 11 with a pair of forceps or the like, or a method of smoothening the inner surface 11 of the urethra by sucking the inner surface 11 using an suction tool and applying tensile force to the inner surface 11, can be employed.

Next, the hemostatic step S14 will be described.

The hemostatic step S14 is a step performed when hemorrhage occurs in the treated site due to the treatment performed in the treatment step S12. As described above, in the treatment step S12, an operation of making an incision in the scar tissue 31 or an operation of scraping the scar tissue 31 is performed. At this time, if the treatment is performed to a depth reaching the corpus spongiosum penis 13, blood flowing in the caverns of corpus spongiosum 14 running in the corpus spongiosum penis 13 may flow out. When hemorrhage has occurred, if temporary hemostasis is performed, strain imposed on a biological body can be reduced and the following steps can be performed without delay.

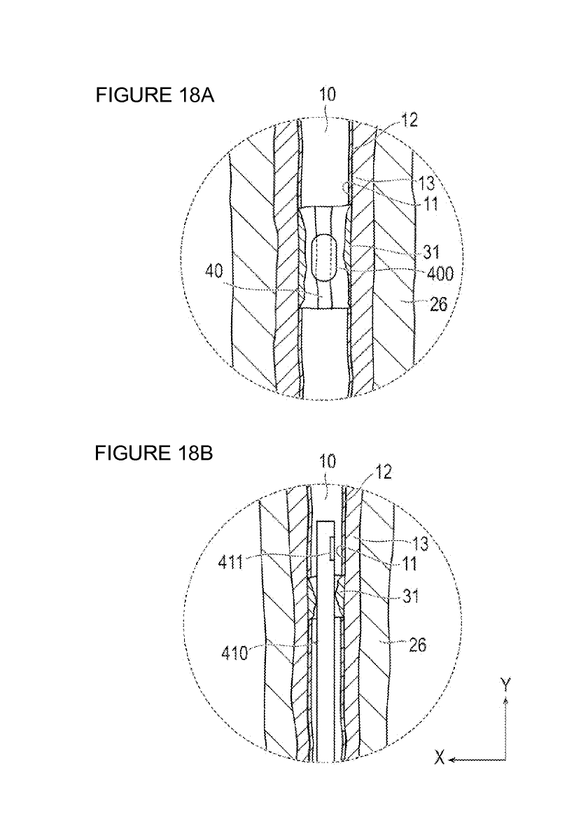

FIG. 18A shows a state in which hemostasis is performed by using a predetermined hemostatic tool 400.

The hemostatic tool 400 is constituted with an adsorptive member that can adsorb blood. As the adsorptive member, for example, a collagen sponge can be used. When the collagen sponge is used, it can also be utilized as a foundation onto which epithelial cells are engrafted. Accordingly, engraftment of epithelial cells in the treatment portion 40 can be suitably promoted. Moreover, for example, the hemostatic tool 400 can be constituted with a porous member having a function of adsorbing blood or with an adsorptive material consisting of a plurality of fibers.

FIG. 18B shows an example of how to use a hemostatic tool 410 according to Modification example 1.

The hemostatic tool 410 can include a sheath that can eb inserted into the urethra 10. In the hemostatic tool 410, a window portion 411 is formed. For example, through the window portion 411, a predetermined treatment tool can be disposed in a position close to the scar tissue 31. Moreover, through the window portion 411, the state or position of the treated site can be confirmed by using a predetermined imaging apparatus or the like. As shown in the drawing, if the site of the hemostatic tool 410 excluding the window portion 411 is pushed and pressed against the hemorrhage site, hemorrhage can be temporarily stopped.

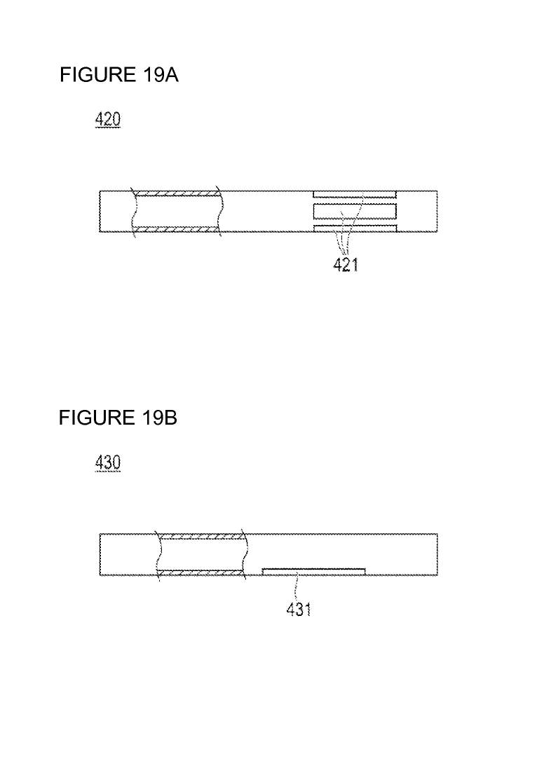

FIG. 19A shows a hemostatic tool 420 according to Modification example 2.

The hemostatic tool 420 is constituted with a sheath that can be inserted into the urethra 10. In the hemostatic tool 420, a plurality of window portions 421 is formed in the circumferential direction of the hemostatic tool 420 around the axis thereof. For example, the hemostatic tool 420 can be used concurrently with a treatment tool such as the treatment tool 150 shown in FIG. 12B that includes a plurality of blade portions 154a to 154c. For example, after an incision is made by the blade portions 154a to 154c protruding from the respective window portions 421, hemostasis can be performed by a method of rotating the hemostatic tool 420 so as to cause the position of the window portions 421 to deviate from the hemorrhage site and then pushing the sheath against the hemorrhage site or by a method of closing the window portions 421 by using other members and then pushing the sheath against the hemorrhage site.

FIG. 19B shows a hemostatic tool 430 according to Modification example 3.

The hemostatic tool 430 is constituted with a sheath that can be inserted into the urethra 10. A window portion 431 is formed at the proximal end side of the sheath 430. As shown in the present modification example, the window portion formed in the sheath used as a hemostatic tool can be disposed in any position.

For example, each of the hemostatic tools 410 to 430 can be used in combination with each of the aforementioned treatment tools. Hereinafter, an example of how to combine the treatment tool 190 shown in FIG. 14 with the hemostatic tool 420 and how to use the combination will be described.

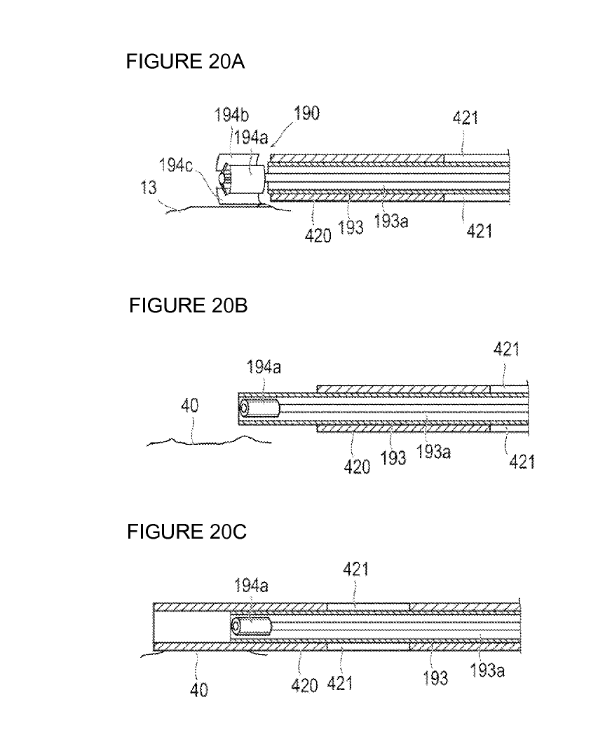

As shown in FIG. 20A, the second sheath 197 of the treatment tool 190 is replaced with the hemostatic tool 420. As shown in FIG. 20B, after the scar tissue 31 is treated using the treatment tool 190, and thus the treatment portion 40 is formed, the respective blade portions 194a to 194c of the treatment tool 190 are accommodated in the lumen 193a of the first sheath 193. When hemorrhage occurs at the time of the treatment performed by the treatment tool 190, as shown in FIG. 20C, hemostasis can be performed by pushing the site of the hemostatic tool 420 excluding the window portion 421 against the hemorrhage site. In this way, if each of the treatment tools is used in combination with each of the hemostatic tools, urethral stricture can be more effectively treated.

Each of the hemostatic tools illustrated in the drawing is an example, and hemostasis can be performed by methods other than the method using each of the illustrated hemostatic tools. For example, hemostasis can be performed by a method of stopping hemorrhage by pressing a portion as a target of hemostasis by using a balloon or the like that can be deformed by dilation, a method of stopping hemorrhage by using an elastic tube that can be deformed according to the shape of the inner surface 11 of the urethra, or a method of suppressing hemorrhage by pressing the tissue in the vicinity of a site as a target of hemostasis by suction or the like.

Next, an insertion step S15 and an assisting step S16 will be described.

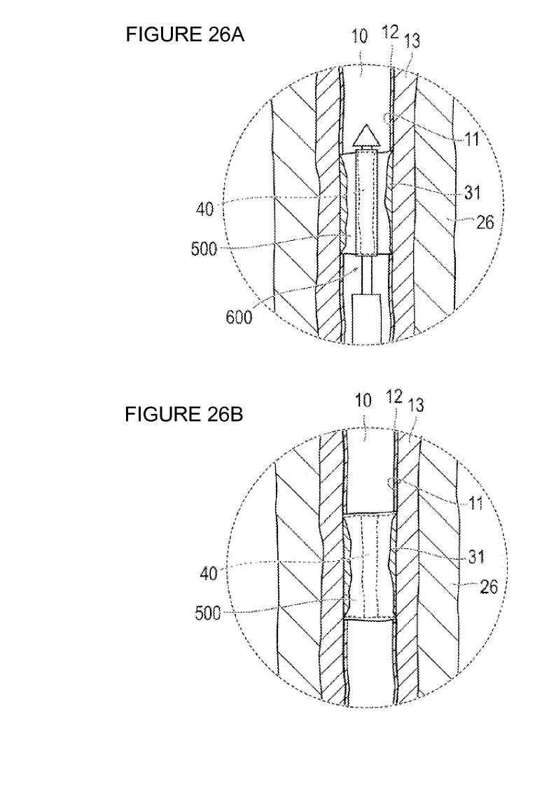

As shown in FIGS. 26A and 26B, the insertion step S15 is a step of transurethrally inserting predetermined assisting means 500 into the urethra 10. Moreover, the assisting step S16 is a step of imparting an epithelial function to the treatment portion 40 by using the assisting means 500 inserted into the urethra 10. As described above, in the urethral stricture treatment method of the prior art, after incision or excision is performed on the scar tissue 31, the treated site is exposed to liquid such as urine, thus the scar tissue 31 becomes thicker, and this can lead to a problem of recurrence of urethral stricture. In the treatment method according to the present embodiment, after the treatment portion 40 is formed, it is protected from liquid (urine or inflammatory components), other internal secretions of a biological body, various germs, and the like by using the assisting means 500. Herein, “imparting an epithelial function by using the assisting means” can include, for example, reconstruction or engraftment of epithelial cells by using oral mucosa (epithelium) as described later and prevention of contact between the treatment portion 40 and liquid or the like.

In order to protect the treatment portion 40 in the urethra 10, the assisting means 500 needs to be guided to the treatment portion 40 through the urethra 10 having a limited cross-sectional area. Therefore, as the assisting means 500, it is preferable to use an object that can be deformed (shaped) so as to have a size, a shape, or the like which enables the object to move in the urethra 10. Moreover, at least a function of preventing the treatment portion 40 from being in contact with liquid or the like is imparted to the assisting means 500 such that it can protect the treatment portion 40. From the viewpoint described above, in the present embodiment, epithelium that is collected from a biological body and processed in the form of a sheet is used as the assisting means 500. The epithelium may be collected from any site in a biological body, but is preferably oral mucosa. The oral mucosa contains epithelial cells, and when the epithelial cell are engrafted into the treatment portion 40, the epithelial function is imparted to the treatment portion 40 (the treatment portion 40 obtains the epithelial function). If the epithelial cells (urethral mucosa) that consistently prevent infiltration of urine are formed, the treatment portion 40 can be protected from liquid such as urine.

The assisting means 500 preferably has flexibility such that it can be reversibly deformed into a spread planar shape from a folded or wound shape. Furthermore, the external shape of the assisting means 500 and the dimension (size, thickness, or the like) of each portion of assisting means 500 can be optionally set in consideration of the size of the treatment portion 40.

Specifically, the insertion step S15 according to the present embodiment can include a step of transurethrally inserting the assisting means 500, which has been shaped to have a first shape that enables the assisting means 500 to be inserted into the urethra 10, into the urethra 10, and a step of disposing the assisting means 500 in the treatment portion 40 formed by treating the scar tissue 31.

An insertion tool 600 used in the insertion step S15 will be described.

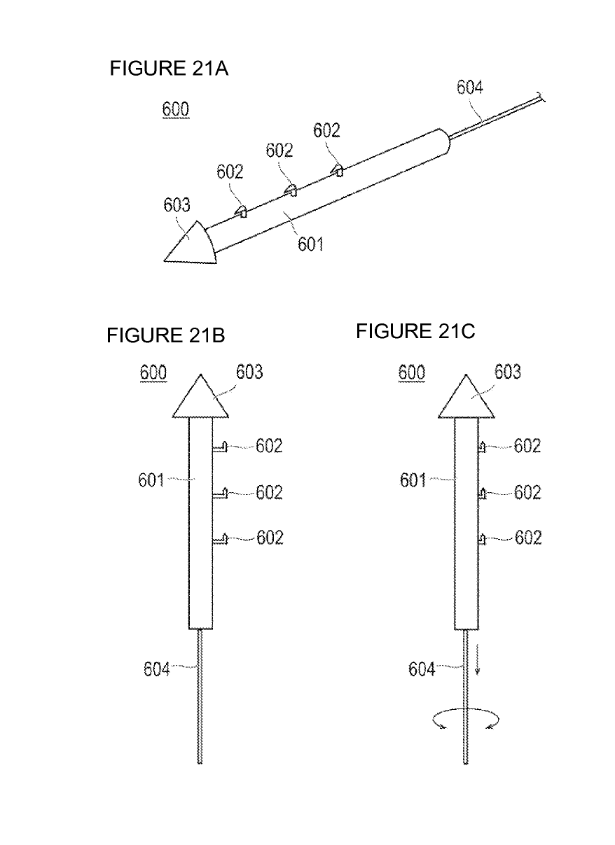

As shown in FIGS. 21A to 21C, the insertion tool 600 can include a main body portion 601 that is in the form of a rod extending in the axial direction, a plurality of holding portions 602 that protrude to the outside from the main body portion 601 in the radial direction, a distal end protecting portion 603 that is disposed at the distal end of the main body portion 601, and an adjusting portion 604 that is provided for adjusting the protrusion amount of the holding portions 602 protruding from the main body portion 601.

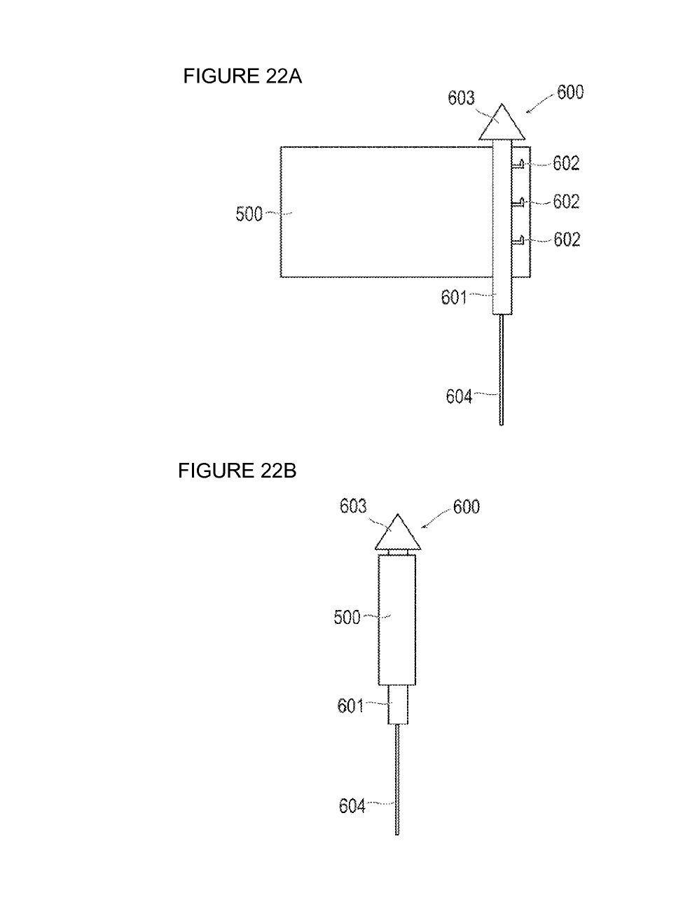

As shown in FIGS. 22A and 22B, the main body portion 601 is a portion around which the assisting means 500 is wound. The holding portion 602 disposed in the main body portion 601 is a portion that can hold the assisting means 500 wound around the main body portion 601 by making the assisting means 500 hooked thereon. By being hooked on the holding portion 602, the assisting means 500 can be prevented from being detached from the insertion tool 600.

The adjusting portion 604 is constituted so as to adjust the protrusion amount of the holding portions 602 by a rotation operation or a pushing and pulling operation. The adjusting portion 604 is connected to the holding portions 602 inside the main body portion 601, though it is not shown in the drawing. As shown in FIG. 21C, when the adjusting portion 604 is rotated, due to the rotation operation, the holding portions 602 are drawn into the main body portion 601. Moreover, when the adjusting portion 604 is pulled, due to the pulling operation, the holding portions 602 are drawn into the main body portion 601. If the protrusion amount of the holding portions 602 is adjusted, the holding portions 602 can be prevented from excessively protruding from the main body portion 601 and the urethra 10 can be prevented from being damaged at the time of using the insertion tool 600.

For example, as shown in FIG. 22B, the assisting means 500 is transurethrally inserted into the urethra 10, in a state of being wounded around the main body portion 601 of the insertion tool 600. If the insertion tool 600 is used, the insertion step S15 can be performed in a state in which the assisting means 500 is shaped to have a cross-sectional shape having a reduced diameter. The distal end protecting portion 603 disposed in the main body portion 601 can prevent the assisting means 500 from being detached from the main body portion 601 by coming into contact with the inner surface 11 of the urethra or the like when being inserted into the urethra 10.

FIG. 26A shows a state in which the assisting means 500 has been inserted into the urethra 10 by using the insertion tool 600. Herein, before the insertion step S15 is performed, a preparation step of detachably mounting the assisting means 500 on the insertion tool 600 is performed according to the aforementioned procedure.

In the preparation step, an operation of winding the assisting means 500 around the main body portion 601 of the insertion tool 600 is performed. After the preparation step ends, the assisting means 500 is inserted into the urethra 10 together with the insertion tool 600. After being moved to the vicinity of the treatment portion 40, the assisting means 500 is spread into a planar shape. As the spreading method, for example, a method of winding the assisting means 500 off the insertion tool 600 by rotating the insertion tool 600 in a direction opposite to the direction in which the assisting means 500 is wound around the insertion tool 600, or a method of detaching the assisting means 500 from the insertion tool 600 by using a pair of forceps or the like can be employed. As shown in FIG. 26B, after the assisting means 500 is spread, the treatment portion 40 is covered with the assisting means 500. As described above, if the assisting means 500 is constituted with epithelium (oral mucosa or the like), by the engraftment of epithelial cells into the treatment portion 40, the epithelial function is imparted. By disposing the assisting means 500 in the treatment portion 40, the assisting step S16 of imparting the epithelial function is performed after the insertion step S15. After the assisting means 500 is disposed in the treatment portion 40, an unnecessary portion of the assisting means 500 may be taken out of the urethra 10 by excision.

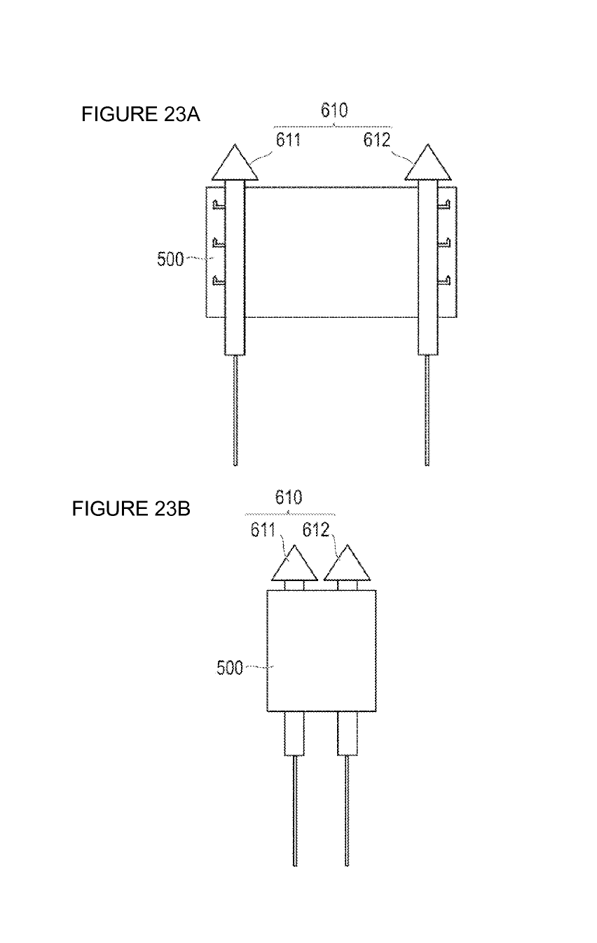

FIGS. 23A and 23B show an example of how to use an insertion tool 610 according to Modification example 1.

The insertion tool 610 is constituted with two insertion tools including a first insertion tool 611 and a second insertion tool 612. Because the constitution of each of the insertion tools 611 and 612 is substantially the same as the constitution of the insertion tool 610 described above, it will not be described. As shown in FIG. 23A, if each of the first insertion tool 611 and the second insertion tool 612 is disposed at both ends of the assisting means 500 and operated such that the assisting means 500 is wound around the insertion tools as shown in FIG. 23B, the assisting means 500 can be simply and rapidly mounted on the insertion tool 610.

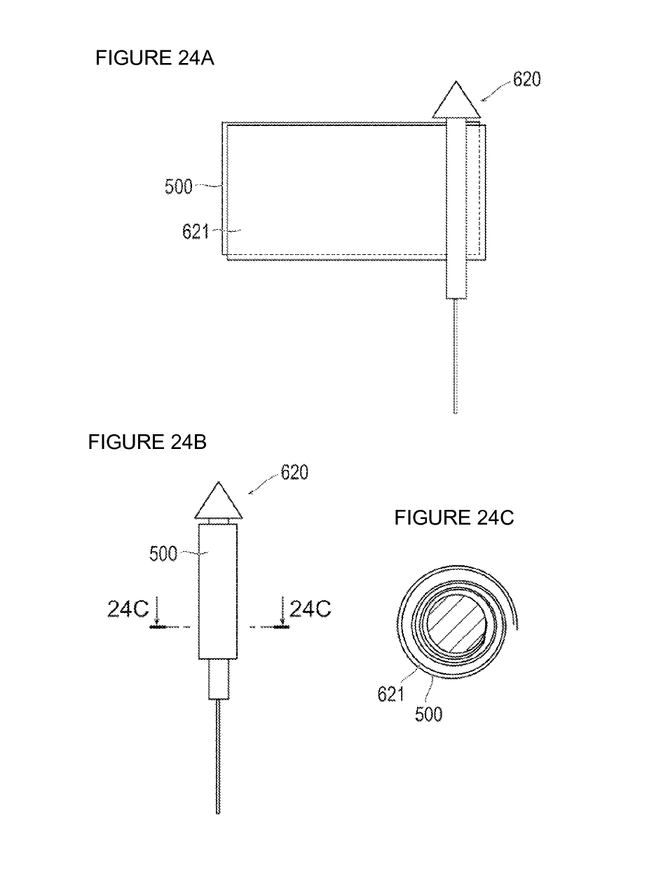

FIGS. 24A to 24C show an example of how to use an insertion tool 620 according to Modification example 2 and an example of how to use a flexible sheet 621 used concurrently with the insertion tool 620. Herein, FIG. 24C is a cross-sectional view taken along line 24C-24C shown in FIG. 24B.

The flexible sheet 621 is used for assisting spreading when the assisting means 500 is spread. As described above, after being inserted into the urethra 10, the assisting means 500 is spread such that it can cover the treatment portion 40. At this time, if resilience of the assisting means 500 is insufficient, the assisting means 500 is not thoroughly spread, and the treatment portion 40 is not easily covered with it. If the flexible sheet 621, which has properties (elasticity and the like) of being able to be smoothly spread in the urethra 10, is wound around the insertion tool 620 beforehand together with the assisting means 500, the spreading of the assisting means 500 can be assisted by the force generated when the flexible sheet 621 is spread. The material, shape, thickness, and the like of the flexible sheet 621 are not particularly limited, as long as the sheet 621 can assist the spreading of the assisting means 500. Furthermore, any of the assisting means 500 and the flexible sheet 621 can be disposed in the inside (outside). However, if the assisting means 500 is constituted with epithelium (oral mucosa or the like), it is preferable that the assisting means 500 is disposed on the outside of the flexible sheet 621.

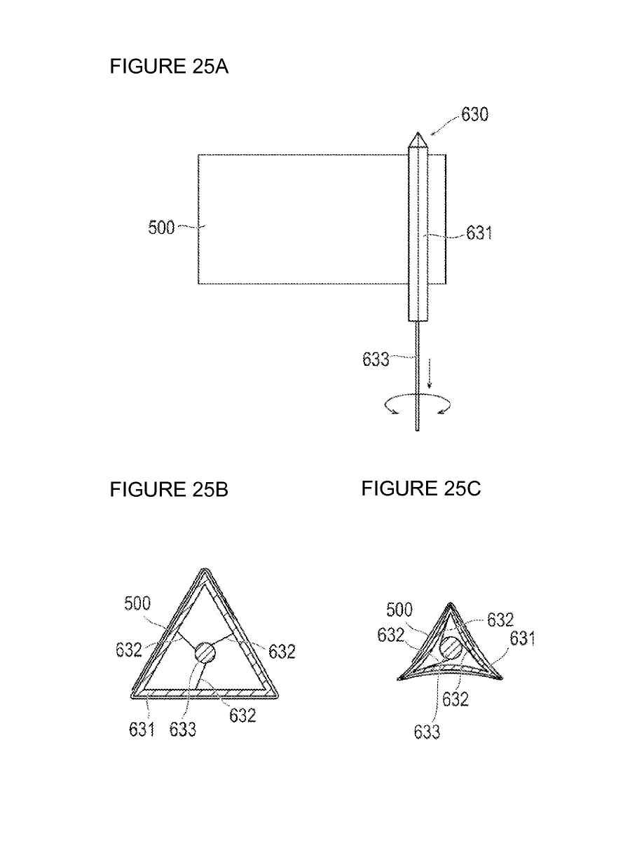

FIGS. 25A to 25C show an example of how to use an insertion tool 630 according to Modification example 3.

The insertion tool 630 can include a main body portion 631 in which the cross section in a direction orthogonal to the axis thereof is in the form of a triangle, deformation portions 632 that are disposed in the inside of the main body portion 631 and are constituted to be able to dilate and contract, and a deformation operation portion 633 for performing an operation for deforming the deformation portions 632. FIG. 25B is a view showing a cross-sectional view of the insertion tool 630 having not yet been deformed by contraction that is taken along a direction orthogonal to the axis thereof. FIG. 25C is a cross-sectional view of the insertion tool 630 deformed by contraction that is taken along a direction orthogonal to the axis thereof.

Each of the deformation portions 632 is constituted with a wire extending in the main body portion 631. Moreover, the deformation operation portion 633 is constituted with a rod-like member that is accommodated in the main body portion 631 and can perform a rotation operation and a pushing and pulling operation. Each of the deformation portions 632 is fixed to the deformation operation portion 633 inside the main body portion 631. When the deformation operation portion 633 is rotated, due to the rotation operation, the deformation portions 632 are wound around the deformation operation portion 633, and thus the size of the main body portion 631 is reduced as shown in FIG. 25C. Furthermore, when the deformation operation portion 633 is pulled, due to the pulling operation, each of the deformation portions 632 and the main body portion 631 are pulled and miniaturized. If the insertion tool 630 is used, the insertion step S15 can be performed in a state in which the insertion tool has been further miniaturized and becomes a compact tool. Accordingly, the operation can be relatively simply and rapidly performed. Herein, for example, by further rounding the main body portion 631, which has the miniaturized umbrella-like shape, in the form of a cylinder, the insertion tool can be constituted to be able to be deformed into a more compact shape.

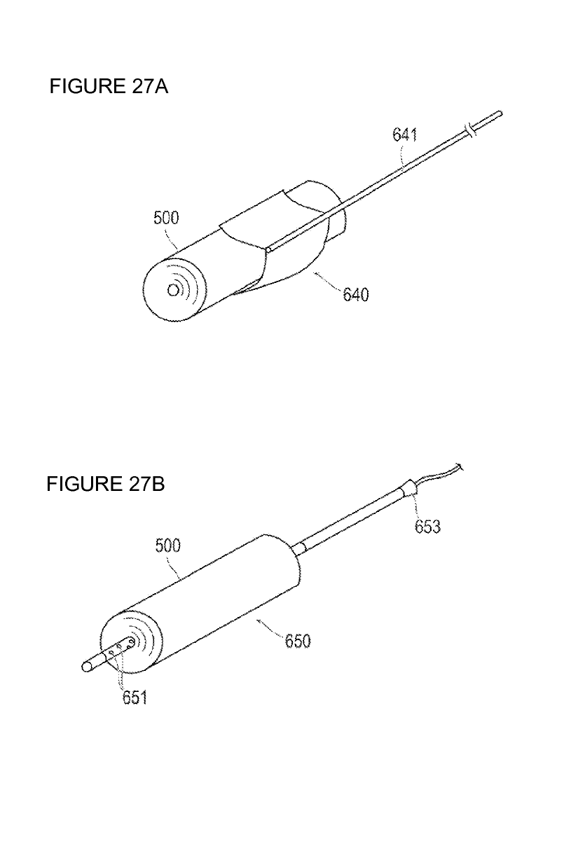

FIG. 27A shows an insertion tool 640 according to Modification example 4.

The insertion tool 640 is constituted with a clip that maintains the shape of the wound assisting means 500 by gripping the wound assisting means 500. Even when the insertion tool 640 is used, the assisting means 500 can be smoothly inserted into the urethra 10. After being inserted into the urethra 10, the assisting means 500 gripped by the insertion tool 640 is released, and in this way, the assisting means 500 can be spread into a shape for covering the treatment portion 40. Moreover, a hand-side operation portion 641 extending to the hand side from the clip portion may be provided, such that the insertion operation and the like into the urethra 10 can be easily performed by the operation at the hand side.

FIG. 27B shows an insertion tool 650 according to Modification example 5.

The insertion tool 650 includes a main body portion 652 in which a plurality of suction holes 651 is formed, and a port 653 which is disposed at the proximal end side of the main body portion 652 and is constituted to be able to be connected to and separated from a predetermined suction device. Inside the main body portion 652, a lumen communicating with each of the suction holes 651 is formed. If air in the lumen of the main body portion 652 is sucked by using a suction tool as an external apparatus, suction force can be applied to the assisting means 500 disposed on the outer surface of the suction holes 651. As a result, the assisting means 500 is held in a state of being wound as shown in the drawing. Furthermore, if the assisting means 500 is inserted into the urethra 10 in a state of being held in this way, and then suction is stopped, the assisting means 500 can be simply spread.

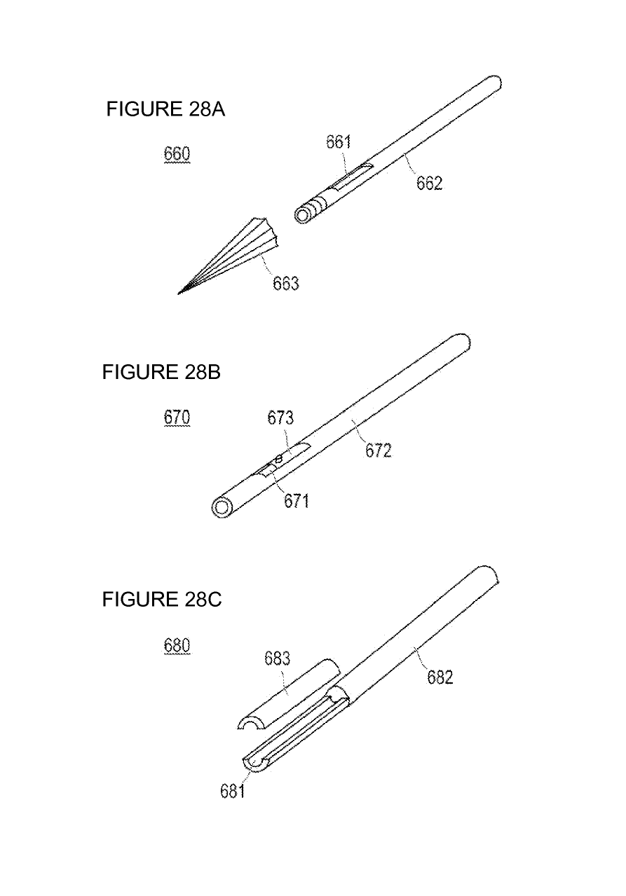

FIG. 28A shows an insertion tool 660 according to Modification example 6.

The insertion tool 660 includes a main body portion 662 in which an opened window portion 661 is formed, and a distal end protecting portion 663 which is constituted to be able to be attached to and detached from the distal end of the main body portion 662. The assisting means 500 shaped by winding or the like can be accommodated in the main body portion 662 through the window portion 661. If the assisting means 500 is accommodated in the main body portion 662, the assisting means 500 can be prevented from being accidently spread. When the insertion tool 660 is inserted into and moved in the urethra 10, the distal end protecting portion 663 enables the insertion tool 660 to smoothly move by reducing sliding resistance.

FIG. 28B shows an insertion tool 670 according to Modification example 7.

The insertion tool 670 can include a main body portion 672 in which an opened window portion 671 is formed, and a sliding door 673 which is provided to open and close the window portion 671. The assisting means 500 shaped by winding or the like can be accommodated in the main body portion 672 through the window portion 671. Furthermore, if the sliding door 673 is moved along the axial direction of the main body portion 672, the window portion 671 can be opened and closed. If the assisting means 500 is inserted into the urethra 10 in a state in which the window portion 671 is closed, the assisting means 500 can be prevented from being accidentally spread.

FIG. 28C shows an insertion tool 680 according to Modification example 8.

The insertion tool 680 can include a main body portion 682 having an opening portion 681 which is formed by removing half of the upper portion of main body portion 682 at the distal end side, and a lid member 683. For example, the lid member 683 is constituted such that it can be freely attached to and detached from the main body portion 682 by fitting or the like. The assisting means 500 shaped by winding or the like can be accommodated in the main body portion 683 through the opening portion 681. Furthermore, if the lid member 683 is mounted on the main body portion 682, the opening portion 681 can be closed. If the assisting means 500 is inserted into the urethra 10 in a state in which the opening portion 681 is closed, the assisting means 500 can be prevented from being accidentally spread.

Each of the insertion tools illustrated in the drawings is an example, and insertion of the assisting means can be performed by methods other than the methods using the illustrated insertion tools. For example, the assisting means can be inserted by a method of miniaturizing a balloon of a balloon catheter (for example, a balloon catheter used for salpingoscopic salpingoplasty) known in the medical field and inserting the assisting means 500 in a state in which it is mounted on the outer surface of the balloon, a method of introducing the assisting means 500 in a state in which it is mounted on the outer circumferential surface of a simple cylindrical member (a polygonal cylinder, a circular cylinder, or the like), or a method of inserting the assisting means 500 by using a guide wire, a pair of forceps, or the like. Moreover, the operation of mounting (including fixing) the sheet-like assisting means 500 on each of the insertion tools may be performed according to procedure in which the insertion tool is loaded on a predetermined worktable and then covered with the assisting means 500 from above, or procedure in which the assisting means 500 is loaded on a worktable, and the insertion tool disposed on the assisting means 500 is operated to rotate. Furthermore, the assisting means 500 to be inserted may have a shape that enables the assisting means 500 to move in the urethra 10. For example, the assisting means 500 may have a folded shape or a rounded shape.

In addition, in the insertion step S15, an imaging apparatus such as an endoscope can be used. In this case, the assisting means 500 may be inserted through a working channel of the imaging apparatus. Moreover, in order to prevent the assisting means 500 from being detached from the insertion tool while moving in the working channel or to prevent deformation of the assisting means 500, it is preferable to concurrently use the insertion tools shown in FIGS. 28A to 28C and the like. Furthermore, if the assisting means 500 is mounted on an endoscope, the endoscope can be used as an insertion tool. In this case, in order to prevent the assisting means 500 from being accidentally detached or spread when the endoscope is inserted into the urethra 10, it can be preferable to mount the distal end protecting portion (head cover) or the like shown in FIG. 28A on the endoscope. In addition, for disposing the assisting means 500 in the treatment portion 40, for example, a positioning operation may be performed by using a measurement instrument having calibrations or the like. When it is difficult to position the assisting means 500 in the treatment portion 40 due to hemorrhage or the like in the treatment step S12, an operation for securing a clear view may be performed by conducting suction or perfusion in the urethra 10.

Up to now, the case of using epithelium as the assisting means has been described. However, the assisting means is not limited to epithelium, and various means having a function of preventing the treatment portion 40 from coming into contact with liquid (urine or the like) can be used. For example, the treatment portion 40 can be protected by being covered with a balloon of a urethral catheter known in the medical field. Alternatively, a sheath or an elastic tube that can be disposed to cover the treatment portion 40, a stent covered with a membranous material that is not infiltrated by liquid, or the like can be used as the assisting means so as to protect the treatment portion 40 over a predetermined period, and in this way, the epithelial function can be secured. For example, a hydrophobic substrate such as Vaseline® (petroleum jelly), a sealing material formed of urethane or a silicone material, a patch, and the like may be used. If the above materials are used, the treatment portion 40 can be prevented from coming into contact with liquid (urine or the like).

When the assisting means does not have a function of engrafting epithelial cells, a biocompatible material which can induce or support the regeneration of epithelial cells or a predetermined member (a patch, a sheet material, or the like) on which epithelial cells are fixed or supported is preferably used together with the assisting means such that the epithelial function is imparted (obtained).

Next, the maintenance step S17 will be described.

If the state in which the treatment portion 40 is protected from liquid or a state in which epithelium (assisting means 500) is brought into contact with the treatment portion 40 is maintained over a predetermined period, the reconstruction of epithelial cells (urethral mucosa) in the treatment portion 40 is suitably accelerated. However, after epithelium or the like is disposed in the treatment portion 40, if the epithelium is separated from the treatment portion 40 due to positional deviation or the like, and thus the contact between the treatment portion 40 and the epithelium is not maintained, the epithelial cells may not be reconstructed. The maintenance step S17 is performed to improve reconstruction properties of the epithelial cells by maintaining the contact between the epithelium inserted into the urethra 10 and the treatment portion 40.

Specifically, the maintenance step S17 according to the present embodiment can include a step of maintaining a state in which the treatment portion 40 comes into contact with (positioned in) at least a portion of the epithelium (assisting means 500) by using predetermined maintenance means transurethrally inserted into the urethra 10. Herein, the amount (contact area) of the epithelium (assisting means 500) coming into contact with the treatment portion 40 is not particularly limited, as long as the epithelium can be engrafted into the treatment portion 40 by coming into contact with the treatment portion 40.

The maintenance means used in the maintenance step S17 will be described.

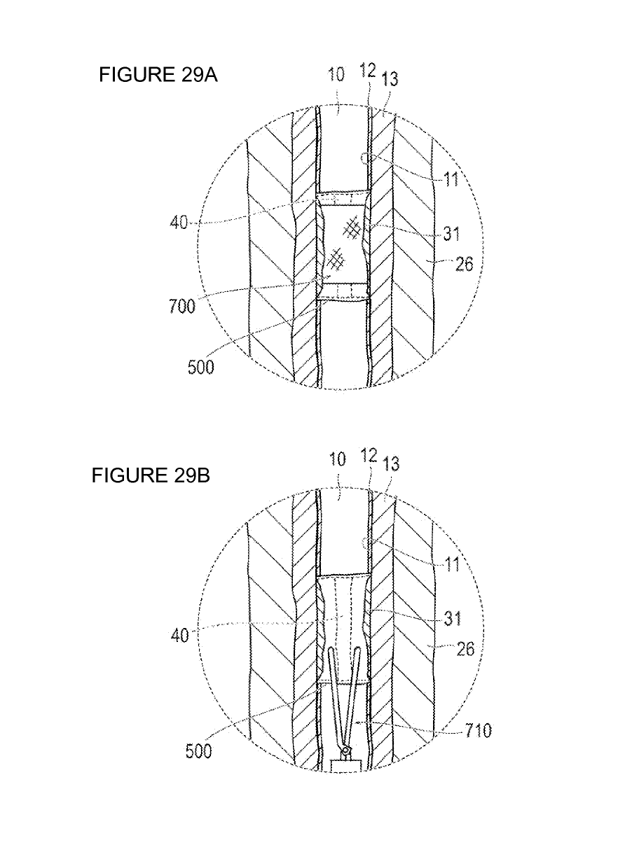

FIG. 29A shows a state in which the maintenance step S17 is performed by maintenance means 700.

The maintenance means 700 is constituted with a hollow stent that is constituted to be able to be deformed by dilation in the urethra 10. The maintenance means 700 is disposed in the inside of the assisting means 500 disposed in a position coming into contact with the treatment portion 40. The maintenance means 700 applies dilation pressure to the assisting means 500. If the assisting means 500 is pushed against the treatment portion 40, the contact between the assisting means 500 and the treatment portion 40 is suitably maintained.

FIG. 29B shows maintenance means 710 according to Modification example 1.

The maintenance means 710 is constituted with a pair of forceps that can be inserted into the urethra 10. If the assisting means 500 is pushed against the treatment portion 40 by the maintenance means 710, the contact between the assisting means 500 and the treatment portion 40 is suitably maintained.

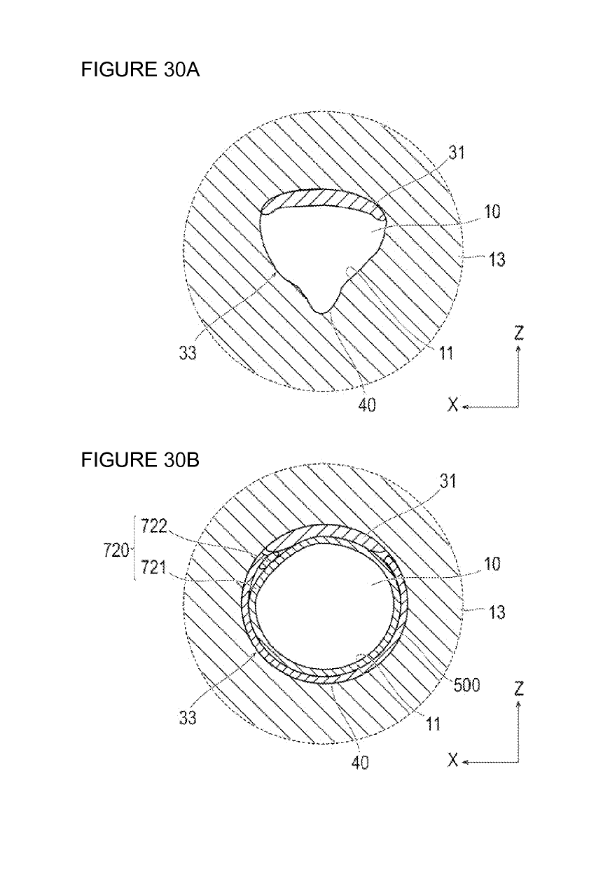

FIG. 30A shows the cross section of the urethra 10 in which the scar tissue 31 has undergone partial incision by the treatment step S12. FIG. 30B shows maintenance means 720 according to Modification example 2. If the maintenance means 720 is used, the insertion step S15 can include a step of disposing the assisting means 500 in a cut 33 formed by incision of the scar tissue 31.

The maintenance means 720 can include a stent 721, which is constituted to be able to dilate in the urethra 10, and a predetermined supporting tool 722, which is used together with the stent 721. By being deformed by dilation, the stent 721 pushes the assisting means 500 against the treatment portion 40, and thus maintains the contact between the assisting means 500 and the treatment portion 40. By receiving the dilation pressure of the stent 721, the urethra 10 is pushed and widens in the radial direction. For example, the supporting tool 722 is disposed so as to fill a void when the void is formed between the stent 722 and the assisting means 500. If the supporting tool 722 is disposed, positional deviation of the assisting means 500 can be prevented by sufficiently applying the dilation pressure of the stent 722 to the assisting means 500. If a void is not formed between the maintenance means 720 and the assisting means 500, the supporting tool 722 may not be used. Moreover, instead of the stent 722, for example, an elastic tube having an outer diameter approximately the same as or slightly greater than the inner diameter of the urethra 10 may be used to prevent the positional deviation of the assisting means 500.

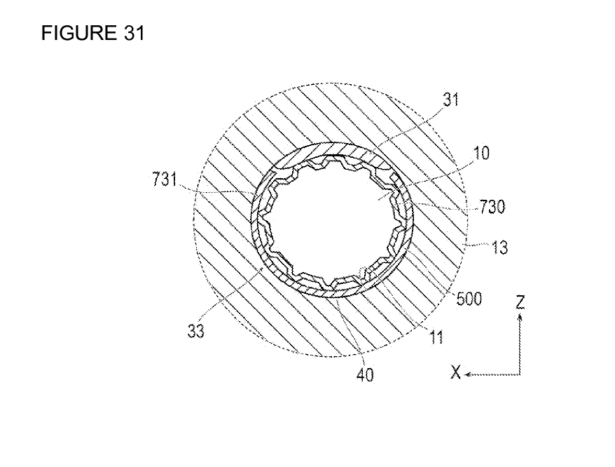

FIG. 31 shows maintenance means 730 according to Modification example 3. If the maintenance means 730 is used, the maintenance step S17 can include a step of bringing at least a portion of the assisting means 500 into contact with the treatment portion 40 by pushing the assisting means 500 against a plurality of different positions in the circumferential direction of the inner surface 11 of the urethra.

The maintenance means 730 is constituted with a stent in which embosses 731 are formed. Due to the dilation pressure of the maintenance means 730, the assisting means 500 can be pushed against the treatment portion 40. Accordingly, the contact between the assisting means 500 and the treatment portion 40 can be suitably maintained. Because a plurality of embosses 731 is formed in the outer circumferential portion of the stent constituted with the maintenance means 730, pressure can be prevented from being applied to the entire circumference of the inner surface 11 of the urethra. Because the use of the stent does not hider blow flow, the urethra can be prevented from being in an ischemic state. The shape or number of the embosses 731 is not limited to those shown in the drawing, and can be appropriately changed. Furthermore, as the maintenance means, an embossed tube which can undergo elastic deformation or a balloon which is constituted to be able to dilate and contract and in which concavities and convexities having the same function as the embosses 731 are formed can be used.

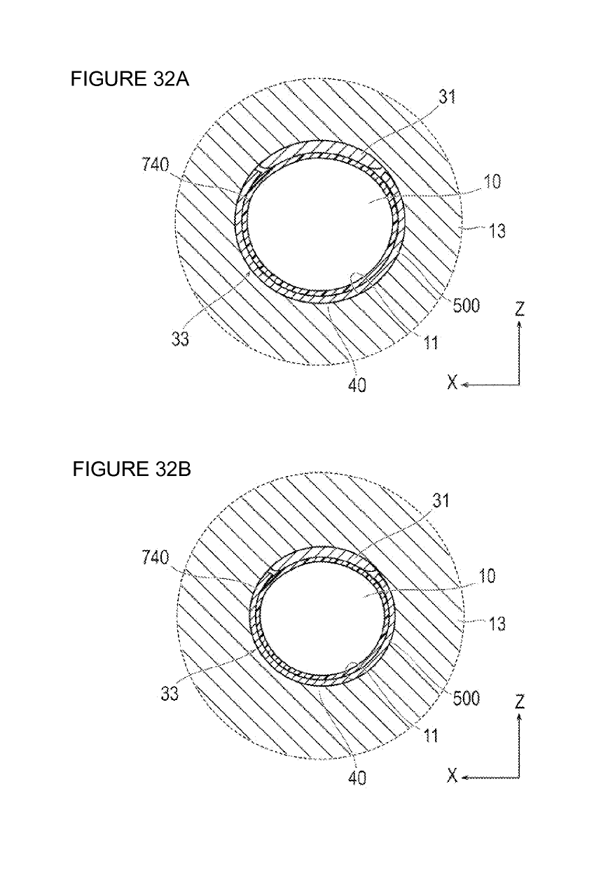

FIGS. 32A and 32B show maintenance means 740 according to Modification example 4. If the maintenance means 740 is used, the maintenance step S17 can include a first pushing step of applying a first pushing force for pushing the assisting means 500 against the treatment portion 40, and a second pushing step of pushing the assisting means 500 against the treatment portion 40 with a pushing force smaller than the first pushing force after the first pushing step.

The maintenance means 740 is constituted with a hollow balloon that can be deformed by dilation and contraction. As shown in FIG. 32A, if the maintenance means 740 is dilated in the urethra 10, the assisting means 500 can be brought into contact with the treatment portion 40 by pushing the assisting means 500 against the treatment portion 40. Moreover, after the engraftment of epithelial cells progresses to a certain extent, if the pushing force applied to the assisting means 500 is reduced by contracting the maintenance means 740 as shown in FIG. 31B, excessive pressure can be prevented from being applied to the inner surface 11 of the urethra and the urethra can be prevented from being in an ischemic state. As the maintenance means 740, for example, a balloon of a urethral balloon catheter can be used. Each of the pushing forces can be optionally set in consideration of the progress of the engraftment of epithelial cells in the treatment portion 40. In addition, the pushing force can be adjusted in two or more stages.

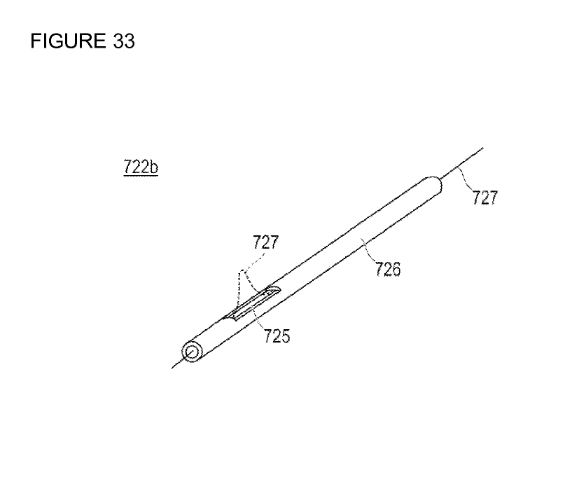

FIG. 33 shows a modification example of the supporting tool 722 shown in FIG. 30B.

A supporting tool 722b according to the modification example can include a sheath 726 in which a window portion 725 is formed, and a wire 727 which is constituted to be able to be deformed so as to protrude from the window portion 725. The wire 727 is shaped such that the portion protruding from the window portion 725 is curved before treatment. If the operation of pushing and pulling the proximal end portion of the wire 727 is performed, the wire 727 can protrude from the window portion 725 and can be stored into the sheath 726. As described above, when a void is formed between a stent or a tube inserted into the urethra 10 and the assisting means 500, if the window portion 725 of the supporting tool 722b is positioned in the void, and then the wire 727 is caused to protrude from the window portion 725 and is pushed against the assisting means 500, positional deviation of the assisting means 500 can be prevented.

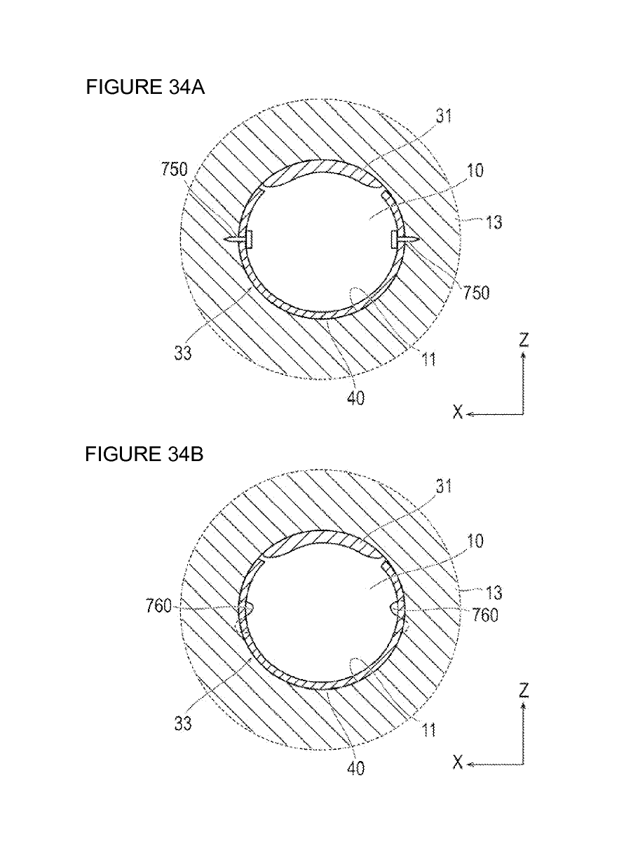

FIG. 34A shows maintenance means 750 according to Modification example 5. The drawing is a cross-sectional view illustrating a state in which the urethra 10 has been dilated by perfusion performed in the urethra 10.

The maintenance means 750 is constituted with a rivet for fixing the assisting means 500. If the maintenance means 750 is used, positional deviation of the assisting means 500 can be prevented and the contact between the assisting means 500 and the treatment portion 40 can be suitably maintained. Herein, the site to which the assisting means 500 is fixed by the maintenance means 750 may be the corpus spongiosum penis 13 or the scar tissue 31.

FIG. 34B shows a maintenance means 760 according to Modification example 6. The drawing is a cross-sectional view illustrating a state in which the urethra 10 has been dilated by perfusion performed in the urethra 10.

The maintenance means 760 is constituted with a suture for stitching the assisting means 500. If the maintenance means 760 is used, the positional deviation of the assisting means 500 can be prevented and the contact between the assisting means 500 and the treatment portion 40 can be suitably maintained. The number of stitches of suturing is not particularly limited as long as the assisting means 500 can be fixed. Herein, the site to which the assisting means 500 is fixed by the maintenance means 760 may be the corpus spongiosum penis 13 or the scar tissue 31.

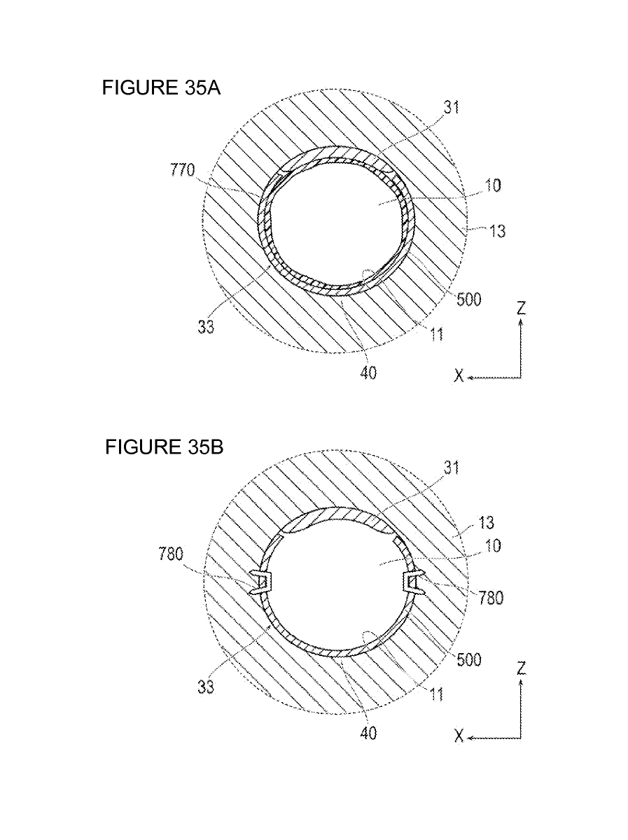

FIG. 35A shows maintenance means 770 according to Modification example 7. The drawing is a cross-sectional view illustrating a state in which the urethra 10 has been dilated by perfusion performed in the urethra 10.

The maintenance means 770 is constituted with a biocompatible adhesive for fixing the assisting means 500 to the scar tissue 31 and the like. If the maintenance means 770 is applied to the surface of the assisting means 500, and the assisting means 500 is fixed to the scar tissue 31, the positional deviation of the assisting means 500 can be prevented and the contact between the assisting means 500 and the treatment portion 40 can be suitably maintained. As the adhesive, for example, an adhesive containing fibrin or the like as a main component can be used. Moreover, for example, a substrate such as a hydrophobic medicine or Vaseline, a sealing material formed of urethane or a silicone material, or a patch can be used. If these materials are used, the treatment portion 40 while fixing the assisting means 500 can be protected.

FIG. 35B shows maintenance means 780 according to Modification example 8. The drawing is a cross-sectional view illustrating a state in which the urethra 10 has been dilated by perfusion performed in the urethra 10.

The maintenance means 780 is constituted with a stapler for fixing the assisting means 500. If the maintenance means 780 is used, the positional deviation of the assisting means 500 can be prevented and the contact between the assisting means 500 and the treatment portion 40 can be suitably maintained. Herein, the site to which the assisting means 500 is fixed by the maintenance means 780 may be the corpus spongiosum penis 13 or the scar tissue 31.

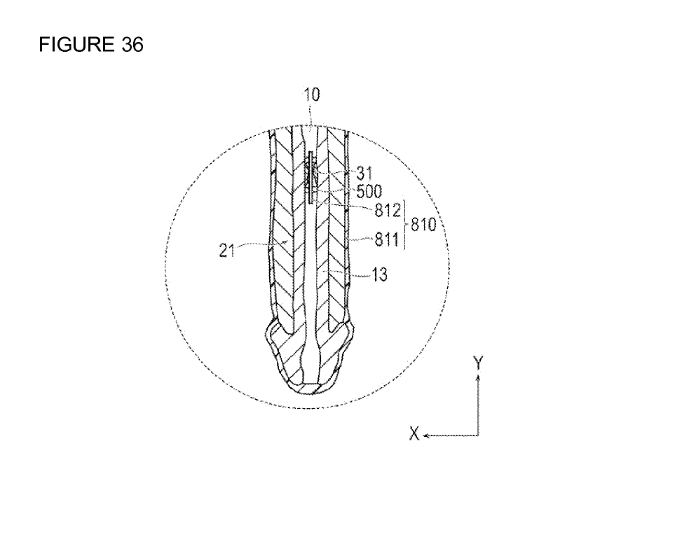

FIG. 36 shows maintenance means 810 according to Modification example 9.

The maintenance means 810 can include an elastic member 811 for covering the penis 21 and supporting means 812 that is disposed in the urethra 10 so as to support the assisting means 500. If pushing force is applied from the outside of the penis 21 by the elastic member 811, and in this state, the assisting means 500 is supported by the supporting means 812 from the inside of the urethra 10, the contact between the assisting means 500 and the treatment portion 40 can be suitably maintained. Herein, the supporting means 812 is constituted with a simple cylindrical member. However, for example, a balloon, a stent, an elastic tube, or a sheath may also be used.

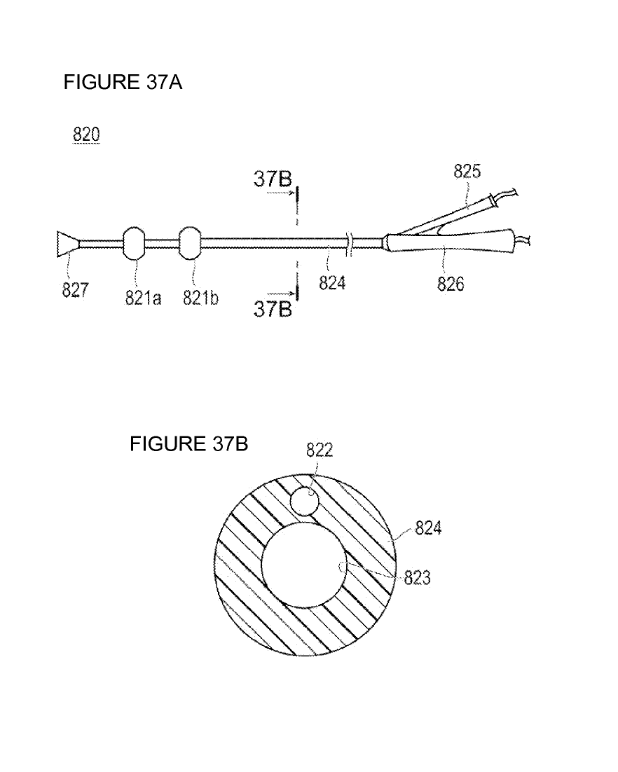

FIGS. 37A and 37B show maintenance means 820 according to Modification example 10. FIG. 37B is a cross-sectional view taken along line 37B-37B shown in FIG. 37A.

The maintenance means 820 can include a first balloon 821a and a second balloon 821b that are arranged in the axial direction in a state of being separated from each other at an interval, a main body portion 824 that is provided with a first lumen 822 communicating with each of the balloons 821a and 821b and a second lumen 823 through which liquid such as urine can circulate, a hub 826 that is provided with a port 825 constituted to be able to be connected to a predetermined tube for supplying a pressure medium to be pumped into each of the balloons 821a and 821b, and a guiding potion 827 that has a tapered shape guiding liquid such as urine into the second lumen 823.

An example of how to use the maintenance means 820 will be described with reference to FIG. 37.

The first balloon 821a blocks liquid such as urine at the distal end side of the second balloon 821b and thus can prevent the liquid from coming into contact with the treatment portion 40. By the guiding portion 827 disposed at the distal end, the liquid such urine is guided to the second lumen 823 disposed in the inside of the main body portion 824 and then discharged to the outside of the biological body. The second balloon 821b dilates in the vicinity of the treatment portion 40, and due to the dilation pressure, the assisting means 500 is pushed against the treatment portion 40. Because the movement of the assisting means 500 is prevented, the contact between the assisting means 500 and the treatment portion 40 can suitably be maintained.

Each of the maintenance means illustrated in the drawings is an example, and the maintenance step S17 can be performed by methods other than the methods using each of the illustrated maintenance means. For example, a shape-memory stent or a shape-memory tube that is formed such that the outer diameter and thickness of the stent or tube can be changed with the passage of time can be used. If the maintenance means is constituted such that it maintains the contact between the assisting means and the treatment portion by applying a relatively high pressure immediately after being inserted into the urethra and then reduces the pressure over time, the urethra can be prevented from being in an ischemic state. In addition, for example, a balloon having micro pores which discharges a pressure medium over time such that the volume of the balloon is reduced over time, a dilative object of which hardness is reduced in response to the change in body temperature (decrease in body temperature), a balloon which is constituted to be able to discharge a pressure medium by an external operation, a balloon using a material which softens when a temperature adjusting medium such as water is supplied thereto from the outside, a mesh-like member or a coil which is constituted or shaped such that it softens or undergoes change of crude density when a temperature adjusting medium such as water is supplied thereto from the outside, or a stent which is constituted to be able to dilate and contract by an external operation can be used. Herein, the maintenance step S17 can be performed by using a single maintenance means or by concurrently using a plurality of any maintenance means. Moreover, if an object (for example, an instrument, a member, or the like such as a balloon that prevents liquid from coming into contact with the treatment portion 40 and is less likely to deviate from the position of the treatment portion 40) other than the sheet-like epithelium is used as the assisting means 500, the maintenance step S17 can be skipped as appropriate.

If each of the steps S11 to S17 described above is performed, the epithelial function can be imparted to the treatment portion 40 and the recurrence of urethral stricture can be suitably prevented. The assisting means 500 is allowed to indwell the urethra 10 over a predetermined period, and after the reconstruction of the epithelial cells in the treatment portion 40 is confirmed, the assisting means 500 is transurethrally taken out of the urethra 10. Moreover, in order to prevent urine or the like flowing in the urethra 10 from hindering each of the steps, urination operation can be continuously or intermittently performed by using a urethral catheter including a known indwelling bladder balloon.

According to the urethral stricture treatment method according to the present embodiment, the treatment portion 40 is formed by performing a predetermined treatment on the scar tissue 31 causing urethral stricture, and then the epithelial function is imparted to the treatment portion 40. In this way, the occurrence of restenosis resulting from thickening of the scar tissue 31 can be prevented. Furthermore, each of the treatment step S12 for forming the treatment portion 40, the confirmation step S13 for confirming whether or not the treatment portion 40 has been formed, and the assisting step S16 of providing the epithelial function to the treatment portion 40 is transurethrally performed. Accordingly, a less invasive procedure greatly reducing strain imposed on a patient can be realized.

Moreover, if the confirmation step S13 includes a step of confirming whether or not at least a portion of the corpus spongiosum penis 13 forming the treatment portion 40 has been exposed from the scar tissue 31 based on an diagnostic image captured by an imaging apparatus, and the treatment of the treatment step S12 is performed until the corpus spongiosum penis 13 is confirmed, the corpus spongiosum penis 13 can be suitably prevented from being unnecessarily damaged in the treatment step S12.

In addition, if the confirmation step S13 includes a step of visually determining whether or not the corpus spongiosum penis 13 has been exposed based on the color change of the diagnostic image, the exposure of the corpus spongiosum penis 13 can be visually confirmed in a relatively simple way and a determination can more accurately be made.

Furthermore, if the treatment step S12 includes a step of making an incision in the scar tissue 31, the treatment portion 40 can be transurethrally formed in a relatively simple and rapid way.

Moreover, if the treatment step S12 includes a step of scraping the scar tissue 31, the treatment portion 40 can be transurethrally formed in a relatively simple and rapid way.

In the treatment step S12, if the treatment is performed plural times while stepwise changing a depth in the thickness direction of the scar tissue 31, the corpus spongiosum penis 13 can be reliably prevented from being unnecessarily damaged during the formation of the treatment portion 40.

Furthermore, if the treatment method further includes a depth diagnosing step of diagnosing a depth of the treatment performed on the scar tissue 31, the treatment can be performed to an appropriate depth. Accordingly, the corpus spongiosum penis 13 can be reliably prevented from being unnecessarily damaged during the formation of the treatment portion 40.

In addition, if the treatment step S12 includes a step of performing a treatment on the scar tissue 31 while avoiding the direction in which the corpus cavernosum penis 26 is positioned in the circumferential direction of the urethra 10, the corpus cavernosum penis 26 can be prevented from being damaged during the formation of the treatment portion 40.

Moreover, if the assisting means 500 is not infiltrated by liquid, the reconstruction of the epithelial function can be suitably assisted in the treatment portion 40 and the occurrence of restenosis can be reliably prevented.

Furthermore, if the assisting means 500 includes epithelium collected from a biological body, epithelial cells can be engrafted into the treatment portion 40. Accordingly, the occurrence of restenosis can be reliably prevented.

The urethral stricture treatment method according to the present invention has been described based on embodiments. However, the present invention is not limited to the description of the embodiments, and can be appropriately modified based on the claims.

In the aforementioned embodiments, the method of performing the respective steps S11 to S17 was described. However, in order to inhibit the recurrence of urethral stricture resulting from restenosis, at least the treatment step S12, the confirmation step S13, and the assisting step S16 may be performed, and other steps can be skipped as appropriate.

The treatment method described based on the embodiments is applicable regardless of the gender of a patient. Furthermore, within the urethra, the treatment method is applicable to any site (for example, a site of urethrae externum side or a site of prostate side).

The detailed description above describes urethral stricture treatment method. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A urethral stricture treatment method comprising:
a treatment step of performing a treatment on scar tissue by using a treatment tool transurethrally inserted into a urethra and forming a treatment portion to which an epithelial function can be imparted;
a confirmation step of confirming whether or not the treatment portion has been formed by using a diagnostic tool transurethrally inserted into the urethra; and
an assisting step of imparting the epithelial function to the treatment portion by using an assisting means transurethrally inserted into the urethra.

2. The urethral stricture treatment method according to claim 1,
wherein the diagnostic tool includes an imaging apparatus that can be transurethrally inserted into the urethra;
the confirmation step includes a step of determining whether or not at least a portion of corpus spongiosum penis forming the treatment portion has been exposed from the scar tissue based on an diagnostic image captured by the imaging apparatus; and
in the treatment step, the treatment is performed until the corpus spongiosum penis is confirmed by the confirmation step.

3. The urethral stricture treatment method according to claim 2,
wherein the confirmation step includes a step of visually determining whether the corpus spongiosum penis has been exposed based on color change of the diagnostic image.

4. The urethral stricture treatment method according to claim 1, wherein the treatment step includes a step of making an incision in the scar tissue.

5. The urethral stricture treatment method according to claim 1, wherein the treatment step includes a step of scraping the scar tissue.

6. The urethral stricture treatment method according to claim 1, wherein the treatment step includes a step of performing a treatment plural times while stepwise changing a depth in a thickness direction of the scar tissue.

7. The urethral stricture treatment method according to claim 1, further comprising:
a depth diagnosing step of diagnosing the depth of the treatment performed on the scar tissue.

8. The urethral stricture treatment method according to claim 1,
wherein the treatment step includes a step of performing the treatment on the scar tissue while avoiding a direction in which a corpus cavernosum penis is positioned in a circumferential direction of the urethra.

9. The urethral stricture treatment method according to claim 1, wherein the assisting means is not infiltrated by liquid.

10. The urethral stricture treatment method according to claim 9, wherein the assisting means includes epithelium collected from a biological body.

11. A method of treating a urethral stricture, the method comprising:
performing a treatment on scar tissue by using a treatment tool transurethrally inserted into a urethra and forming a treatment portion to which an epithelial function can be imparted;
confirming whether or not the treatment portion has been formed by using a diagnostic tool transurethrally inserted into the urethra; and
imparting the epithelial function to the treatment portion by using an assisting means transurethrally inserted into the urethra.

12. The method according to claim 11, comprising:
determining whether at least a portion of corpus spongiosum penis forming the treatment portion has been exposed from the scar tissue based on an diagnostic image captured by an imaging apparatus that can be transurethrally inserted into the urethra; and
performing the treatment until the corpus spongiosum penis is confirmed.

13. The method according to claim 12, comprising:
visually determining whether the corpus spongiosum penis has been exposed based on color change of the diagnostic image.

14. The method according to claim 11, comprising:
making an incision in the scar tissue.

15. The method according to claim 11, comprising:
scraping the scar tissue.

16. The method according to claim 11, comprising:
performing the treatment a plurality of times while stepwise changing a depth in a thickness direction of the scar tissue.

17. The method according to claim 1, comprising:
diagnosing the depth of the treatment performed on the scar tissue.

18. The method according to claim 11, comprising:
performing the treatment on the scar tissue while avoiding a direction in which a corpus cavernosum penis is positioned in a circumferential direction of the urethra.

19. The method according to claim 11, wherein the assisting means is not infiltrated by liquid.

20. The method according to claim 19, wherein the assisting means includes epithelium collected from a biological body.
